(19) United States
(12) Patent Application Publication
(10) Pub. No.: US 2009/0306038 A1
(43) Pub. Date: Dec. 10, 2009

(54) 2-Aminopyrimidine derivatives as modulators of the histamine H4 receptor activity

(76) Inventors: Elena Carceller González, Barcelona (ES); Jorge Salas Solana, Barcelona (ES); Robert Soliva Soliva, Barcelona (ES); Eva Maria Medina Fuentes, Barcelona (ES); Josep Marti Via, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/066,594

(22) PCT Filed: Sep. 12, 2006
(86) PCT No.: PCT/EP06/66303

§ 371 Date: Aug. 29, 2008

(30) Foreign Application Priority Data
Sep. 13, 2005 (EP) 05380195.7
Jun. 9, 2006 (EP) 06381027.9

(51) Int. Cl. A61K 31/506; C07D 403/04; A61K 31/551

(52) U.S. Cl. 514/210.2; 540/575; 514/218; 544/332

(57) ABSTRACT

2-Aminopyrimidine derivatives of formula (I) that are useful as modulators of the H₄ receptor.

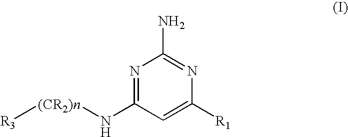

### FIELD OF THE INVENTION

The present invention relates to a new series of 2-aminopyrimidine derivatives, to processes to prepare them, to pharmaceutical compositions comprising these compounds as well as to their use in therapy.

### BACKGROUND OF THE INVENTION

Histamine is one of the most potent mediators of immediate hypersensibility reactions. While histamine effects on muscle contraction, vascular permeability and gastric acid secretion are well known, its effects on the immune system are becoming unveiled.

Recently, a novel histamine receptor, which has been named H₄, has been cloned by several groups working separately. As the other members of its family, it is a G-protein coupled receptor (GPCR) containing 7 transmembrane segments. However, the H₄ receptor has low homology with the three other histamine receptors; it is remarkable that it shares only a 35% amino acid homology with the H₃ receptor. While the expression of the H₃ receptor is restricted to cells of the central nervous system, the expression of the H₄ receptor has been observed in cells of the haematopoietic lineage, in particular eosinophils, mast cells, basophils, dendritic cells and T-cells. The fact that H₄ expression is limited to these specific cell types suggests the involvement of the H₄ receptor in immuno-inflammatory responses. Moreover, this hypothesis is reinforced by the fact that its gene expression can be regulated by inflammatory stimulus such as interferon, TNFα and IL-6. In addition, it has been recently published that the H₄ receptor is expressed in human synovial cells obtained from patients suffering from rheumatoid arthritis.

Recent studies with specific ligands of the H₄ receptor have helped to delimit the pharmacological properties of this receptor. These studies have evidenced that several histamine-induced responses in eosinophils such as chemotaxis, conformational change and CD11b and CD54 up-regulation are mediated specifically by the H₄ receptor. In addition, the role of the H₄ receptor in mast cells has been studied. Although H₄ receptor activation does not induce mast cell degranulation, histamine and other proinflammatory mediators are released. Moreover, calcium mobilization and chemotaxis induction have been also observed. With regard to T-lymphocytes, it has been demonstrated that the IL-16 release from CD8⁺ T is dependent on H₄ receptor.

The various functions of the H₄ receptor observed in eosinophils, mast cells and T-cells therefore suggest that this receptor can play an important role in the immuno-inflammatory responses. In fact, H₄ receptor antagonists have shown activity in murine models of peritonitis, pleurisy and scratching. In addition, in vivo activity has been observed in an experimental model of inflammatory bowel disease.

It is therefore expected that H₄ receptor antagonists can be useful for the treatment or prevention of immunological or inflammatory diseases, including asthma, allergic rhinitis, chronic obstructive pulmonary disease (COPD), allergic rhinoconjunctivitis, cutaneous allergic diseases such as atopic dermatitis and urticaria, inflammatory bowel diseases, rheumatoid arthritis and psoriasis.

Accordingly, it would be desirable to provide novel compounds having high affinity for the H₄ receptor.

### DESCRIPTION OF THE INVENTION

One aspect of the present invention relates to the compounds of formula I

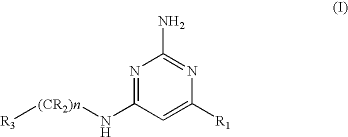

wherein:
R₁ represents a group selected from (a), (b) and (c):

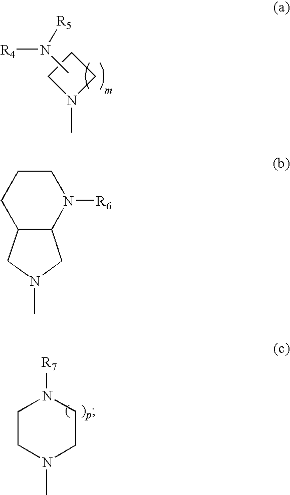

R₂ represents H or C₁₋₄ alkyl;
R₃ represents phenyl optionally fused to a 5- or 6-membered aromatic, saturated or partially unsaturated ring, which can be carbocyclic or heterocyclic with 1 or 2 heteroatoms selected from N, O and S, where R₃ can be optionally substituted with one or more substituents R₈;
R₄ represents H or C₁₋₄ alkyl;
R₅ represents H or C₁₋₄ alkyl;
R₆ represents H or C₁₋₄ alkyl;
R₇ represents H or C₁₋₄ alkyl;
each R₈ independently represents C₁₋₄ alkyl halogen, —OH, C₁₋₄ alkoxy, C₁₋₄ alkylthio, C₁₋₄ haloalkyl, C₁₋₄ haloalkoxy, —COR₉, —CO₂R₉, —CONR₉R₉, —NR₉R₉, —NHCOR₁₀, —CN, C₂₋₄ alkynyl, or —CH₂OH, and additionally one of the substituents R₈ can represent phenyl optionally substituted with one or more groups selected from C₁₋₄ alkyl halogen, —OH, C₁₋₄ alkoxy, C₁₋₄ alkylthio, C₁₋₄ haloalkyl, C₁₋₄ haloalkoxy, —COR₉, —CO₂R₉, —CONR₉R₉, —NR₉R₉, —NHCOR₁₀, —CN, C₂₋₄ alkynyl, and —CH₂OH;
R₉ represents H or C₁₋₄ alkyl;
R₁₀ represents C₁₋₄ alkyl;
m represents 1, 2 or 3;
n represents 0 or 1; and
p represents 1 or 2.

The present invention also relates to the salts and solvates of the compounds of formula I.

Some compounds of formula I can have chiral centres that can give rise to various stereoisomers. The present invention relates to each of these stereoisomers and also mixtures thereof.

The compounds of formula I exhibit high affinity for the H₄ receptor. Thus, another aspect of the invention relates to a compound of general formula I

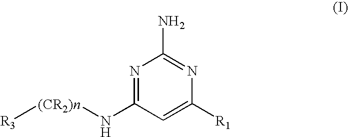

wherein:
R₁ represents a group selected from (a), (b) and (c):

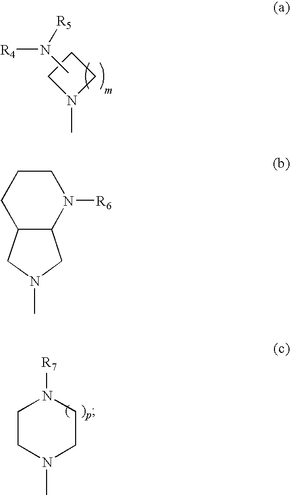

R₂ represents H or C₁₋₄ alkyl;
R₃ represents phenyl optionally fused to a 5- or 6-membered aromatic, saturated or partially unsaturated ring, which can be carbocyclic or heterocyclic with 1 or 2 heteroatoms selected from N, O and S, where R₃ can be optionally substituted with one or more substituents R₈;
R₄ represents H or C₁₋₄ alkyl;
R₅ represents H or C₁₋₄ alkyl;
R₆ represents H or C₁₋₄ alkyl;
R₇ represents H or C₁₋₄ alkyl;
each R₈ independently represents C₁₋₄ alkyl halogen, —OH, C₁₋₄ alkoxy, C₁₋₄ alkylthio, C₁₋₄ haloalkyl, C₁₋₄ haloalkoxy, —COR₉, —CO₂R₉, —CONR₉R₉, —NR₉R₉, —NHCOR₁₀, —CN, C₂₋₄ alkynyl, or —CH₂OH, and additionally one of the substituents R₈ can represent phenyl optionally substituted with one or more groups selected from C₁₋₄ alkyl halogen, —OH, C₁₋₄ alkoxy, C₁₋₄ alkylthio, C₁₋₄ haloalkyl, C₁₋₄ haloalkoxy, —COR₉, —CO₂R₉, —CONR₉R₉, —NR₉R₉, —NHCOR₁₀, —CN, C₂₋₄ alkynyl, and —CH₂OH;
R₉ represents H or C₁₋₄ alkyl;
R₁₀ represents C₁₋₄ alkyl;
m represents 1, 2 or 3;
n represents 0 or 1; and
p represents 1 or 2;
for use in therapy.

Another aspect of this invention relates to a pharmaceutical composition which comprises a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment or prevention of diseases mediated by the histamine H₄ receptor.

Another aspect of the present invention relates to the use of a compound of formula I or a pharmaceutically acceptable salt thereof for the manufacture of a medicament for the treatment or prevention of immunological or inflammatory diseases.

Another aspect of the present invention relates to the use of a compound of formula I or a pharmaceutically acceptable salt thereof for the manufacture of a medicament for the treatment or prevention of a disease selected from asthma, allergic rhinitis, chronic obstructive pulmonary disease (COPD), allergic rhinoconjunctivitis, cutaneous allergic diseases, inflammatory bowel diseases, rheumatoid arthritis and psoriasis.

Another aspect of the present invention relates to the use of a compound of formula I or a pharmaceutically acceptable salt thereof for the treatment or prevention of diseases mediated by the histamine H₄ receptor.

Another aspect of the present invention relates to the use of a compound of formula I or a pharmaceutically acceptable salt thereof for the treatment or prevention of immunological or inflammatory diseases.

Another aspect of the present invention relates to the use of a compound of formula I or a pharmaceutically acceptable salt thereof for the treatment or prevention of a disease selected from asthma, allergic rhinitis, chronic obstructive pulmonary disease (COPD), allergic rhinoconjunctivitis, cutaneous allergic diseases, inflammatory bowel diseases, rheumatoid arthritis and psoriasis.

Another aspect of the present invention relates to a method of treating or preventing a disease mediated by the histamine H₄ receptor in a subject in need thereof, especially a human being, which comprises administering to said subject a therapeutically effective amount of a compound of formula I or a pharmaceutically acceptable salt thereof.

Another aspect of the present invention relates to a method of treating or preventing immunological or inflammatory diseases in a subject in need thereof, especially a human being, which comprises administering to said subject a therapeutically effective amount of a compound of formula I or a pharmaceutically acceptable salt thereof.

Another aspect of the present invention relates to a method of treating or preventing a disease selected from asthma, allergic rhinitis, chronic obstructive pulmonary disease (COPD), allergic rhinoconjunctivitis, cutaneous allergic diseases, inflammatory bowel diseases, rheumatoid arthritis and psoriasis, in a subject in need thereof, especially a human being, which comprises administering to said subject a therapeutically effective amount of a compound of formula I or a pharmaceutically acceptable salt thereof.

Another aspect of the present invention relates to a process for the preparation of a compound of formula I as defined above, which comprises:

(a) reacting a compound of formula II, or a salt thereof, with a compound of formula III

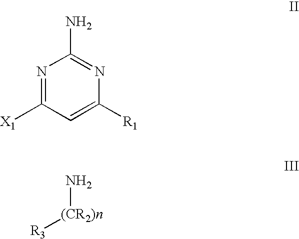

wherein R₁, R₂, R₃ and n have the meaning described above and X₁ represents halogen; or
(b) reacting a compound of formula IV, or a salt thereof, with a compound of formula V

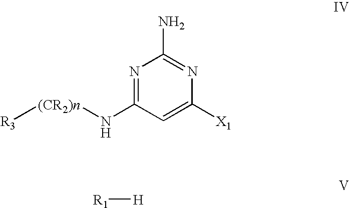

wherein R₁, R₂, R₃ and n have the meaning described above and X₁ represents halogen; or
(c) converting, in one or a plurality of steps, a compound of formula I into another compound of formula I.

In the present invention, the term C₁₋₄ alkyl means a straight or branched alkyl chain which contains from 1 to 4 carbon atoms. It thus includes the groups methyl, ethyl, propyl, isopropyl, butyl, isobutyl, sec-butyl and tert-butyl. The term C₁₋₂ alkyl refers to the groups methyl and ethyl.

A C₁₋₄ haloalkyl group means a group resulting from the replacement of one or more hydrogen atoms from a C₁₋₄ alkyl group with one or more halogen atoms (i.e. fluoro, chloro, bromo or iodo), which can be the same or different. Examples include, among others, trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl and nonafluorobutyl.

A C₁₋₄ alkoxy group means an alkoxy group having from 1 to 4 carbon atoms, the alkyl moiety having the same meaning as previously defined. Examples include methoxy, ethoxy, propoxy, isopropoxy, butoxy, isobutoxy, sec-butoxy and tert-butoxy.

A C₁₋₄ alkylthio group (i.e. —S—C₁₋₄ alkyl) means an alkylthio group having from 1 to 4 carbon atoms, the alkyl moiety having the same meaning as previously defined. Examples include methylthio, ethylthio, propylthio, isopropylthio, butylthio, isobutylthio, sec-butylthio and tert-butylthio.

A C₁₋₄ haloalkoxy group means a group resulting from the replacement of one or more hydrogen atoms from a C₁₋₄ alkoxy group with one or more halogen atoms (i.e. fluoro, chloro, bromo or iodo), which can be the same or different. Examples include, among others, trifluoromethoxy, fluoromethoxy, 1-chloroethoxy, 2-chloroethoxy, 1-fluoroethoxy, 2-fluoroethoxy, 2-bromoethoxy, 2-iodoethoxy, 2,2,2-trifluoroethoxy, pentafluoroethoxy, 3-fluoropropoxy, 3-chloropropoxy, 2,2,3,3-tetrafluoropropoxy, 2,2,3,3,3-pentafluoropropoxy, heptafluoropropoxy, 4-fluorobutoxy and nonafluorobutoxy.

A C₂₋₄ alkynyl group means a straight or branched alkyl chain which contains from 2 to 4 carbon atoms and that also contains one or two triple bonds. Examples include, among others, the groups ethynyl, 1-propynyl, 2-propynyl, 1-butynyl, 2-butynyl, 3-butynyl and 1,3-butadiynyl.

A halogen radical means fluoro, chloro, bromo or iodo.

In a compound of formula I, R₃ represents a phenyl group which optionally can be fused to a 5- or 6-membered ring which can be aromatic, saturated or partially unsaturated. This ring to which the phenyl is fused (“fused ring”) can be carbocyclic or heterocyclic, in which case it may contain 1 or 2 heteroatoms independently selected from N, O and S. Moreover, when the fused ring is not aromatic, one or more C ring atoms can be optionally oxidized to form CO groups. Examples of R₃ when the phenyl group is fused to a carbocyclic ring with the features defined above include naphthyl, indanyl, tetrahydro-naphthyl, 1H-indenyl, 1-oxo-4H-naphthyl, 1-oxoindenyl, 3,4-dihydro-1-oxo-2H-naphthyl and 1-oxoindanyl. Examples of R₃ when the phenyl group is fused to a heterocyclic ring with the features defined above include, among others, indolyl, benzofuryl, benzo[b]thienyl, quinolinyl, isoquinolinyl, 3-dihydrobenzoxazolyl, 2,3-dihydrobenzothiazolyl, 1H-benzimidazolinyl, 2,3-dihydro-1H-isoindolyl, 2,3-dihydro-1H-indolyl, benzoxazolyl, benzoxathiazolyl, 1H-indazolyl, quinoxalinyl, 1,4-dihydroquinoxalinyl, quinazolinyl, phtalazinyl, 1,4-dihydroquinazolinyl, isochromanyl, 1H-isochromenyl, 4H-chromenyl, 2,3-dihydrobenzofuryl, 2,3-dihydrobenzo[b]thienyl, 1,2-dihydroquinolinyl, 1,2,3,4-tetrahydroquinolinyl, 1,2-dihydroisoquinolinyl, 1,2,3,4-tetrahydroisoquinolinyl, 3,4-dihydrobenzo[c][1,2]dioxinyl, 4H-benzo[1,3]dioxinyl, 3H-benzo[1,2]dioxolyl, benzo[1,3]dioxolyl, 3,4-dihydro-2H-benzo[1,4]oxazinyl, 1,2,3,4-tetrahydroquinoxalinyl, 4-oxo-1H-quinazolinyl, 4-oxo-1H-quinolinyl, 2-oxo-1,3-dihydroindolyl and 4-oxa-2,3-dihydro-1H-quinolinyl.

The expression “optionally substituted with one or more” means that a group can be substituted with one or more, preferably with 1, 2, 3 or 4 substituents, more preferably with 1 or 2 substituents, provided that said group has enough positions available susceptible of being substituted. When present, these substituents can be the same or different, and they can be placed on any available position.

In a compound of formula I, the R₃ group can be optionally substituted with one or more R₈ groups, as mentioned above. The R₈ groups can be the same or different and can be placed on any available position of the R₃ group, that is, they can be placed on either the phenyl ring or the fused ring when R₃ is a phenyl fused to a second ring.

In a group R₁ of formula (a), the amino substituent of formula —NR₄R₅ can be placed on any available position of the cyclic amine with the exception of the carbon atoms adjacent to the ring N atom.

The invention thus relates to the compounds of formula I as defined here above.

In another embodiment, the invention relates to compounds of formula I wherein n is 0.

In another embodiment, the invention relates to compounds of formula I wherein R₂ represents H or methyl.

In another embodiment, the invention relates to compounds of formula I wherein R₃ represents phenyl or naphthyl, which can be optionally substituted with one or more substituents R₈.

In another embodiment, the invention relates to compounds of formula I wherein R₃ represents phenyl optionally substituted with one or more substituents R₈.

In another embodiment, the invention relates to compounds of formula I wherein each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl.

In another embodiment, the invention relates to compounds of formula I wherein each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl.

In another embodiment, the invention relates to compounds of formula I wherein R₃ represents phenyl or naphthyl, which can be optionally substituted with one or more substituents R₈; and

each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl.

In another embodiment, the invention relates to compounds of formula I wherein R₃ represents phenyl or naphthyl, which can be optionally substituted with one or more substituents R₈;

each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl; and

n is 0.

In another embodiment, the invention relates to compounds of formula I wherein R₃ represents phenyl optionally substituted with one or more substituents R₈;

each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl; and

n is 0.

In another embodiment, the invention relates to compounds of formula I wherein R₃ represents phenyl optionally substituted with one or more substituents R₈;

each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl; and

n is 0.

In another embodiment, the invention relates to compounds of formula I wherein R₁ represents (a) or (b).

In another embodiment, the invention relates to compounds of formula I wherein R₁ represents (a).

In another embodiment, the invention relates to compounds of formula I wherein R₁ represents (b).

In another embodiment, the invention relates to compounds of formula I wherein R₁ represents (c).

In another embodiment, the invention relates to compounds of formula I wherein m represents 1 or 2.

In another embodiment, the invention relates to compounds of formula I wherein p represents 2.

In another embodiment, the invention relates to compounds of formula I wherein m represents 1 or 2, and p represents 2.

In another embodiment, the invention relates to compounds of formula I wherein R₄ represents H or C₁₋₂ alkyl.

In another embodiment, the invention relates to compounds of formula I wherein R₅ represents H or C₁₋₂ alkyl.

In another embodiment, the invention relates to compounds of formula I wherein R₄ is H and R₅ is methyl or ethyl, or R₄ and R₅ are H, or R₄ and R₅ are methyl.

In another embodiment, the invention relates to compounds of formula I wherein R₆ is H or methyl.

In another embodiment, the invention relates to compounds of formula I wherein R₇ is H or methyl.

In another embodiment, the invention relates to compounds of formula I wherein R₁ represents (a) or (b) and m represents 1 or 2.

In another embodiment, the invention relates to compounds of formula I wherein R₁ represents (a) and m represents 1 or 2.

In another embodiment, the invention relates to compounds of formula I wherein R₁ represents (a), m represents 1 or 2, R₄ represents H or C₁₋₂ alkyl and R₅ represents H or C₁₋₂ alkyl.

In another embodiment, the invention relates to compounds of formula I wherein R₁ represents (b) and R₆ represents H or methyl.

In another embodiment, the invention relates to compounds of formula I wherein R₁ represents (c) and p represents 2.

In another embodiment, the invention relates to compounds of formula I wherein R₁ represents (c), p represents 2 and R₇ is H or methyl.

In another embodiment, the invention relates to compounds of formula I wherein:

R₁ represents (a), (b) or (c);
m represents 1 or 2;
p represents 2;
R₃ represents phenyl optionally substituted with one or more substituents R₈;
each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl; and
n is 0.

In another embodiment, the invention relates to compounds of formula I

wherein:
R₁ represents (a) or (b);
m represents 1 or 2;
R₃ represents phenyl optionally substituted with one or more substituents R₈;
each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl; and
n is 0.

In another embodiment, the invention relates to compounds of formula I wherein R₁ represents (a);

m represents 1 or 2;
R₃ represents phenyl optionally substituted with one or more substituents R₈;
each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl; and
n is 0.

In another embodiment, the invention relates to compounds of formula I wherein R₁ represents (a) or (b), and n is 0.

In another embodiment, the invention relates to compounds of formula I wherein R₁ represents (a) or (b), R₄ is H and R₅ is methyl or ethyl.

In another embodiment, the invention relates to compounds of formula I wherein R₁ represents (a) or (b), and R₄ and R₅ are H.

In another embodiment, the invention relates to compounds of formula I wherein R₁ represents (a) or (b), and R₄ and R₅ are methyl.

In another embodiment, the invention relates to compounds of formula I wherein R₁ represents (a) or (b), and R₆ is H or methyl.

In another embodiment, the invention relates to compounds of formula I wherein R₁ represents (a) or (b), and R₃ represents phenyl or naphthyl, which can be optionally substituted with one or more substituents R₈.

In another embodiment, the invention relates to compounds of formula I wherein R₁ represents (a) or (b), and R₃ represents phenyl, which can be optionally substituted with one or more substituents R₈.

In another embodiment, the invention relates to compounds of formula I wherein:

R₁ represents (a) or (b);
R₃ represents phenyl or naphthyl, which can be optionally substituted with one or more substituents R₈; and
each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl.

In another embodiment, the invention relates to compounds of formula I wherein:

R₁ represents (a) or (b);
R₃ represents phenyl optionally substituted with one or more substituents R₈;
each R₈ independently represents C₁₋₄ alkyl, halogen, —OH, C₁₋₄ alkoxy, C₁₋₄ haloalkyl, C₁₋₄ haloalkoxy, —CN or C₂₋₄ alkynyl, and additionally one of the substituents R₈ can represent phenyl optionally substituted with one or more groups selected from C₁₋₄ alkyl, halogen, —OH, C₁₋₄ alkoxy, C₁₋₄ haloalkyl, C₁₋₄ haloalkoxy, —CN and C₂₋₄ alkynyl; and
n is 0.

In another embodiment, the invention relates to compounds of formula I wherein R₁ represents (c) and n is 0.

In another embodiment, the invention relates to compounds of formula I wherein R₁ represents (c) and n is 1.

In another embodiment, the invention relates to compounds of formula I wherein R₁ represents (c) and p is 2.

In another embodiment, the invention relates to compounds of formula I wherein R₁ represents (c) and p is 1.

In another embodiment, the invention relates to compounds of formula I wherein R₁ represents (c) and R₃ represents phenyl optionally substituted with one or more substituents R₈.

In another embodiment, the invention relates to compounds of formula I wherein R₁ represents (c) and R₇ is H or methyl.

In another embodiment, the invention relates to compounds of formula I wherein R₁ represents (c) and R₂ is H.

Furthermore, the present invention covers all possible combinations of particular and preferred groups described hereinabove.

In a further embodiment, the invention relates to a compound of formula I selected from the list of examples 1 to 202.

In a further embodiment, the invention relates to compounds according to formula I which provide more than 50% inhibition of H₄ receptor activity at 1 μM, more preferably at 0.1 μM in a H₄ receptor binding assay such as the one described in example 203.

The compounds of the present invention may contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when they are used for therapeutic purposes. The term pharmaceutically acceptable salt represents those salts which are, according to medical judgement, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in the conventional manner. The salts of the compounds of formula I can be converted into other salts of the compounds of formula I by ion exchange using ion exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof) and a solvent. Examples of solvents include pharmaceutically acceptable solvents such as water, ethanol and the like. A complex with water is known as a hydrate. Solvates of compounds of the invention (or salts thereof), including hydrates, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of general formula I. Optically pure isomers can also be individually obtained using enantiospecific synthesis. The present invention covers all individual isomers as well as mixtures thereof (for example racemic mixtures or mixtures of diastereomers), whether obtained by synthesis or by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups by conventional protective groups, particularly when amino groups are present. Both the nature of these protective groups and the procedures for their introduction or removal are well known in the art (see for example Greene T. W. and Wuts P. G. M, “Protective Groups in Organic Synthesis”, John Wiley & Sons, 3^{rd} edition, 1999). Whenever a protecting group is present, a subsequent step for removing said protecting group may be required, which is carried out in the standard conditions. As an example, as protective groups of an amino function the groups tert-butoxycarbonyl (Boc) or benzyl (Bn) can be used, or else the amino group can be protected in the form of a 2,5-dimethyl-1H-pyrrol-1-yl group.

Unless otherwise stated, in the methods described below the meanings of the different substituents are the meanings described above with regard to a compound of formula I.

In general, the compounds of formula I can be obtained by reacting a compound of formula II, or a salt thereof, with a compound of formula III, as shown in the following scheme:

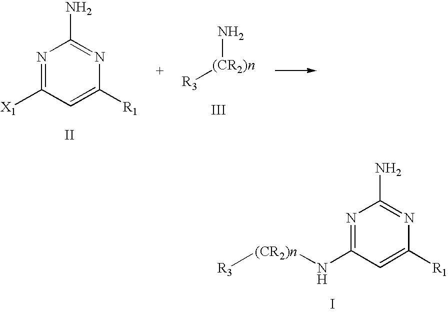

wherein R₁, R₂, R₃ and n have the meaning described above in connection with a compound of general formula I and X₁ represents halogen, preferably chloro. The amino substituents of the compounds of formula II are usually protected to avoid the formation of side products.

The reaction can be carried out by heating at a suitable temperature, for example at a temperature comprised between 70° C. and 190° C., preferably at a temperature comprised between 120° C. and 170° C. Optionally, the reaction can be carried out by using microwaves irradiation at a wattage that allows to reach these temperatures. The reaction can be carried out without solvent or in a suitable solvent such as ethanol, methanol or butanol. When in the compounds of formula I n is 0, the reaction can be carried out in the presence of an acid, such as hydrochloric acid.

The compounds of formula I wherein n=0 are preferably obtained starting from a salt of the amine of formula II, preferably the hydrochloride, in a suitable solvent such as ethanol, methanol or butanol.

The compounds of formula I wherein n=0 can alternatively be obtained in the presence of a palladium catalyst, including for instance, palladium diacetate, a phosphine ligand, preferably 2,2′-bis(diphenylphosphino)-1,1′-binaphthyl (BINAP), and a base, preferably sodium tert-butoxide. The reaction may be carried out in a solvent such as dioxane, 1,2-dimethoxyethane or N,N-dimethylformamide, and preferably in toluene. The reaction can be carried out by heating at a suitable temperature comprised between 20° C. and 120° C. The NH₂ group of the compounds of formula II must be conveniently protected to perform the palladium-catalyzed reaction.

The compounds of formula II can be obtained by reacting a compound of formula VI with a compound of formula V, as shown in the following scheme:

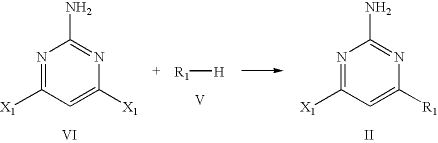

wherein R₁ has the meaning described above and X₁ represents halogen, preferably chloro. The reaction can be carried out in the presence of a base, including organic amines such as pyridine, triethylamine, N,N-ethyldiisopropylamine, dimethylaniline and diethylaniline among others, in a suitable solvent such as ethanol, methanol or butanol, and heating, preferably at reflux. The amino substituents of the compounds of formula V are usually protected to conduct the reaction.

The compounds of formula III are either commercially available or can be obtained by methods described in the literature. Compounds of formula V and VI are commercially available or are readily obtained from commercially available compounds by standard procedures.

Alternatively, the compounds of formula I can be obtained by reacting a compound of formula IV, or a salt thereof, with a compound of formula V, as shown in the following scheme:

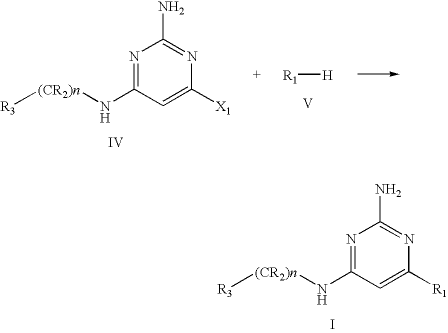

wherein R₁, R₂, R₃ and n have the meaning described above in connection with a compound of general formula I, and X₁ represents halogen, preferably chloro.

The reaction can be carried out in the presence of a base, including organic amines such as pyridine, triethylamine, N,N-ethyldiisopropylamine, dimethylaniline and diethylaniline among others, and heating at a suitable temperature comprised between 80° C. and 120° C. in a suitable solvent such as ethanol, methanol or butanol.

The compounds of formula IV can be obtained by reacting a compound of formula VI with a compound of formula III, as shown in the following scheme:

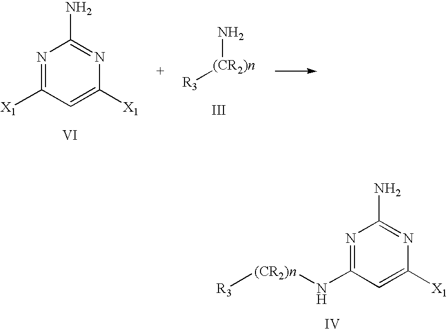

wherein R₂, R₃ and n have the meaning described above and X₁ represents halogen, preferably chloro. The reaction can be carried out in the presence of a base, including organic amines such as pyridine, triethylamine, N,N-ethyldiisopropylamine, dimethylaniline and diethylaniline among others, in a suitable solvent, preferably dioxane, and heating, preferably at reflux.

Moreover, certain compounds of the present invention can also be obtained starting from other compounds of formula I by appropriate conversion reactions of functional groups in one or several steps, using well-known reactions in organic chemistry under the reported standard experimental conditions.

As previously mentioned, the compounds of the present invention show high affinity for the histamine H₄ receptor. Therefore, the compounds of the invention are expected to be useful to treat or prevent diseases mediated by the H₄ receptor in mammals, including human beings.

Diseases that can be treated or prevented with the compounds of the present invention include among others immunological or inflammatory diseases such as asthma, allergic rhinitis, chronic obstructive pulmonary disease (COPD), allergic rhinoconjunctivitis, cutaneous allergic diseases (such as atopic dermatitis and urticaria), inflammatory bowel diseases (such as ulcerative colitis and Crohn's disease), rheumatoid arthritis and psoriasis.

Assays to determine the ability of a compound to interact with the histamine H₄ receptor are well known in the art. For example, one can use a H₄ receptor binding assay such as the one explained in detail in example 203. Another useful assay is a GTP [γ-³⁵S] binding assay to membranes that express the H₄ receptor. Functional assays can also be carried out with H₄ receptor-expressing cells, in a system measuring any kind of cellular activity mediated by a second messenger associated with H₄, such as intracellular cAMP levels or Ca²⁺ mobilization.

For selecting active compounds, testing at 1 μM must result in an activity of more than 50% inhibition in the test provided in example 203. More preferably, compounds should exhibit more than 50% inhibition at 0.1 μM.

The present invention also relates to a pharmaceutical composition which comprises a compound of the present invention (or a pharmaceutically acceptable salt or solvate thereof) and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the addition of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as purified water, ethanol, sorbitol, glycerol, polyethylene glycols (macrogols) and propylene glycol. Said compositions can also contain coadjuvants such as wetting, suspending, sweetening, flavouring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions which will be dissolved in water or any other sterile injectable medium immediately before use. It is also possible to start from sterile materials and keep them under these conditions throughout all the manufacturing process.

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as a single or divided doses.

The invention is illustrated by the following examples.

### EXAMPLES

The following abbreviations have been used in the examples:

AcN: acetonitrile
AcOEt: ethyl acetate
BINAP: 2,2′-bis(diphenylphosphino)-1,1′-binaphthyl
n-BuOH: 1-butanol

### DIEA: N,N-Ethyldiisopropylamine

EtI: ethyl iodide
Et₃N: triethylamine
EtOH: ethanol
MeI: methyl iodide
MeOH: methanol
Na^{t}BuO: sodium tert-butoxide
Pd(OAc)₂: palladium diacetate
THF: tetrahydrofuran
t_{R}: retention time
LC-MS: liquid chromatography-mass spectrometry

LC-MS spectra have been performed using the following chromatographic methods:

Method 1: Column X-Terra, MS C18 5 μm (100 mm×2.1 mm), temperature: 30° C., flow: 0.35 mL/min, eluent: A=AcN, B=NH₄HCO₃ 10 mM, gradient: 0 min 10% A; 10 min 90% A; 15 min 90% A; 15.01 min 10% A.

Method 2: Column X-bridge, MS C18 2.5 μm (50 mm×2.1 mm), temperature: 50° C., flow: 0.50 mL/min, eluent: A=NH₄HCO₃ 10 mM, B=AcN, C=H₂O, gradient: 0 min 10% A, 10% B; 4 min 10% A, 85% B; 4.75 min 10% A, 85% B; 4.76 min 10% A, 10% B.

Method 3: Column X-bridge, MS C18 2.5 μm (50 mm×2.1 mm), temperature: 30° C., flow: 0.35 mL/min, eluent: A=AcN, B=0.1% HCO₂H, gradient: 0 min 10% A; 10 min 90% A; 15 min 90% A; 15.01 min 10% A.

### Reference Example 1

### 2-Amino-4-chloro-6-(4-methyl-[1,4]diazepan-1-yl)pyrimidine

To a solution of 2-amino-4,6-dichloropyrimidine (3 g, 0.018 mmol) and DIEA (4.8 mL, 0.028 mmol) in EtOH (18 mL) under argon atmosphere, 1-methylhomopiperazine was added (2.3 mL, 0.018 mmol) and the resulting mixture was stirred at reflux for 3 hours. It was allowed to cool to room temperature and the solid obtained was filtrated and dried under vacuum for 18 h, to afford 2.33 g of the title compound (yield: 53%).

### Reference Examples 2-4

Following a similar procedure to that described in reference example 1, but using the corresponding starting materials in each case, the following compounds were obtained:

| Reference | | | Method | | m/z |
|---|---|---|---|---|---|
| example | Name | Starting materials | (LC-MS) | t_{R} (min) | (MH⁺) |
| 2 | 2-Amino-4-chloro-6-(4- | 2-amino-4,6- | — | — | — |
| | methylpiperazin-1- | dichloropyrimidine and | | | |
| | yl)pyrimidine | 1-methylpiperazine | | | |
| 3 | tert-Butyl 4-(2-amino-6- | 2-amino-4,6- | 1 | 7.17 | 314 |
| | chloropyrimidin-4- | dichloropyrimidine and | | | |
| | yl)piperazine-1-carboxylate | 1-(tert- | | | |
| | | butoxycarbonyl)piperazine | | | |
| 4 | tert-Butyl 4-(2-amino-6- | 2-amino-4,6- | 1 | 6.80 | 328 |
| | chloropyrimidin-4-yl)- | dichloropyrimidine and | | | |
| | [1,4]diazepane-1- | 1-(tert- | | | |
| | carboxylate | butoxycarbonyl)homopiperazine | | | |

### Reference Example 5

### tert-Butyl methyl[(3R)-pyrrolidin-3-yl]carbamate

### (a) tert-Butyl[(3R)-1-benzylpyrrolidin-3-yl]methylcarbamate

To a solution of (3R)-1-benzyl-N-methylpyrrolidin-3-amine (10 g, 52.55 mmol) in 115 mL of CH₂Cl₂, cooled at 0° C., ditertbutyl dicarbonate (11.6 g, 53.07 mmol) dissolved in 15 mL of CH₂Cl₂ was added. The resulting solution was stirred at room temperature for 18 hours. The solvent was evaporated and the crude product was chromatographed on silica gel using mixtures of hexane/AcOEt of increasing polarity as eluent, to afford 14.5 g of the title compound (yield: 95%).

LC-MS (Method 1): t_{R}=9.55 min; m/z=291 (MH⁺).

### (b) Title Compound

A solution of the compound obtained above (14.5 g, 50.14 mmol), Pd/C (10%, 50% in water) (3 g) and ammonium formate (12.7 g, 200.5 mmol) in a mixture of MeOH (390 mL) and water (45 mL) was heated at reflux for 5 hours. The reaction was filtered through Celite and the filtrate was washed with AcOEt and MeOH. The solvent was evaporated to dryness to afford 10.6 g of the title compound as an oil (yield: 100%).

¹H NMR (300 MHz, CDCl₃) δ: 1.38 (s, 9H), 1.72 (m, 1H), 1.96 (m, 1H), 2.53 (s, NH), 2.80 (s, 3H), 2.87 (m, 1H), 2.93 (m, 1H), 3.11 (m, 2H), 4.58 (m, 1H).

### Reference Example 6

### tert-Butyl azetidin-3-yl(methyl)carbamate

### (a) tert-Butyl[1-(diphenylmethyl)azetidin-3-yl]methylcarbamate

Following a similar procedure to that described in section a of reference example 5, but using 1-(diphenylmethyl)-N-methylazetidin-3-amine instead of (3R)-1-benzyl-N-methylpyrrolidin-3-amine, the desired compound was obtained with 73% yield.

LC-MS (Method 1): t_{R}=10.14 min; m/z=353 (MH⁺).

### (b) Title Compound

A solution of the compound obtained above (6.18 g, 17.53 mmol) in 60 mL of MeOH and 15 mL of AcOEt was purged with argon. Pd/C (10%, 50% in water) (929 mg) was added and then, the solution was purged again with argon and stirred under H₂ atmosphere for 18 hours. The reaction was filtered through Celite and the filtrate was washed with AcOEt and MeOH. The solvent was evaporated to dryness to afford 5.66 g of a mixture of the title compound together with one equivalent of diphenylmethane, which was further used as obtained.

¹H NMR (300 MHz, CD₃O₃) δ: 1.44 (s, 9H), 2.88 (s, 3H), 3.56 (m, 2H), 3.71 (m, 2H), 4.75 (m, 1H).

### Reference Example 7

### tert-Butyl azetidin-3-yl(ethyl)carbamate

### (a) tert-Butyl[1-(diphenylmethyl)azetidin-3-yl]carbamate

Following a similar procedure to that described in section a of reference example 5, but using 1-(diphenylmethyl)azetidin-3-amine instead of (3R)-1-benzyl-N-methylpyrrolidin-3-amine, the title compound was obtained with 61% yield.

LC-MS (Method 1): t_{R}=9.07 min; m/z=339 (MH⁺).

### (b) tert-Butyl[1-(diphenylmethyl)azetidin-3-yl]ethylcarbamate

To a suspension of 55% NaH (985 mg, 22.5 mmol), THF (40 mL) and EtI (2.34 mL, 28.7 mmol) cooled at 0° C., the compound obtained above was added (6.9 g, 20.5 mmol) and the resulting mixture was stirred at room temperature for 18 h. Then, additional 55% NaH (500 mg, 11.45 mmol) and EtI (1.3 mL, 16.2 mmol) were added and stirred at room temperature for 18 h. Some drops of water were added and the mixture was partitioned between AcOEt and water. The organic phase was dried over Na₂SO₄ and concentrated to dryness. The crude product was chromatographed on silica gel using mixtures of hexane/AcOEt of increasing polarity as eluent, to afford 5.13 g of the desired compound (yield: 68%).

LC-MS (Method 1): t_{R}=10.78 min; m/z=367 (MH⁺).

### (c) Title Compound

Following a similar procedure to that described in section b of reference example 6 but using tert-butyl[1-(diphenylmethyl)azetidin-3-yl]ethylcarbamate instead of tert-butyl[1-(diphenylmethyl)azetidin-3-yl]methylcarbamate, the title compound was obtained with 100% yield.

¹H NMR (300 MHz, CDCl₃) δ (TMS): 1.11 (t, J=7.04 Hz, 3H), 1.45 (s, 9H), 1.81 (s, NH), 3.30 (q, J=7.04 Hz, 2H), 3.67 (m, 2H), 3.73 (m, 2H), 4.69 (m, 1H).

### Reference Example 8

### tert-Butyl[(3R)-1-(2-amino-6-chloropyrimidin-4-yl)pyrrolidin-3-yl]methylcarbamate

To a solution of 2-amino-4,6-dichloropyrimidine (1 g, 6.09 mmol) and DIEA (1.6 mL, 9.1 mmol) in EtOH (8 mL) under argon atmosphere, the compound obtained in reference example 5 was added (1.2 g, 6.09 mmol) and the resulting mixture was stirred at reflux for 3 hours. It was allowed to cool to room temperature, the solid obtained was filtered and the mother liquors were concentrated to dryness. The crude product obtained was purified by chromatography on silica gel using hexane/AcOEt mixtures of increasing polarity as eluent, to afford 1.04 g of the title compound (yield: 52%).

LC-MS (Method 1): t_{R}=7.12 min; m/z=328 (MH⁺).

### Reference Examples 9-17

Following a similar procedure to that described in reference example 8, but using the corresponding starting materials in each case, the following compounds were obtained:

| Reference | | | Method | | m/z |
|---|---|---|---|---|---|
| example | Name | Starting material | (LC-MS) | t_{R} (min) | (MH⁺) |
| 9 | tert-Butyl [1-(2-amino-6- | tert-Butyl | 1 | 7.06 | 328 |
| | chloropyrimidin-4- | methyl[pyrrolidin-3- | | | |
| | yl)pyrrolidin-3- | yl]carbamate | | | |
| | yl]methylcarbamate | | | | |
| 10 | tert-Butyl [(3R)-1-(2-amino- | tert-Butyl [(3R)- | 1 | 6.14 | 314 |
| | 6-chloropyrimidin-4- | pyrrolidin-3-yl]carbamate | | | |
| | yl)pyrrolidin-3-yl]carbamate | | | | |
| 11 | tert-Butyl [1-(2-amino-6- | Reference example 6 | 2 | 2.46 | 314 |
| | chloropyrimidin-4- | | | | |
| | yl)azetidin-3- | | | | |
| | yl]methylcarbamate | | | | |
| 12 | tert-Butyl [1-(2-amino-6- | Reference example 7 | 2 | 2.59 | 328 |
| | chloropyrimidin-4- | | | | |
| | yl)azetidin-3- | | | | |
| | yl]ethylcarbamate | | | | |
| 13 | 4-Chloro-6-[3- | N,N-Dimethylpyrrolidin- | 1 | 4.35 | 242 |
| | (dimethylamino)pyrrolidin-1- | 3-amine | | | |
| | yl]pyrimidin-2-amine | | | | |
| 14 | tert-Butyl [1-(2-amino-6- | tert-Butyl piperidin-3- | 1 | 6.87 | 328 |
| | chloropyrimidin-4- | ylcarbamate | | | |
| | yl)piperidin-3-yl]carbamate | | | | |
| 15 | tert-Butyl [1-(2-amino-6- | tert-Butyl piperidin-4- | 1 | 6.81 | 328 |
| | chloropyrimidin-4- | ylcarbamate | | | |
| | yl)piperidin-4-yl]carbamate | | | | |
| 16 | tert-Butyl 6-(2-amino-6- | tert-Butyl octahydro-1H- | 2 | 2.73 | 354 |
| | chloropyrimidin-4- | pyrrolo[3,4-b]pyridine-1- | | | |
| | yl)octahydro-1H-pyrrolo[3,4- | carboxylate | | | |
| | b]pyridine-1-carboxylate | | | | |
| 17 | 4-Chloro-6-[(3R)-3- | (3R)—N,N- | 1 | 4.64 | 242 |
| | (dimethylamino)pyrrolidin-1- | Dimethylpyrrolidin-3- | | | |
| | yl]pyrimidin-2-amine | amine | | | |

### Reference Example 18

### tert-Butyl[(3S)-1-(2-amino-6-chloropyrimidin-4-yl)pyrrolidin-3-yl]methylcarbamate

Following a similar procedure to that described in reference example 8 but using the corresponding (S)-enantiomer as starting material, which was obtained following a similar procedure as in reference example 5, the desired compound was obtained with 76% yield.

LC-MS (Method 1): t_{R}=7.19 min; m/z=328 (MH⁺).

### Reference Example 19

### tert-Butyl {(3R)-1-[2-(2,5-dimethyl-1H-pyrrol-1-yl)-6-chloropyrimidin-4-yl]pyrrolidin-3-yl}carbamate

### (a) 4,6-Dichloro-2-(2,5-dimethyl-1H-pyrrol-1-yl)pyrimidine

A solution of 2-amino-4,6-dichloropyrimidine (10 g, 60.9 mmol) acetonylacetone (13.9 g, 121 mmol) and p-toluenesulphonic acid (116 mg, 0.6 mmol) in toluene (300 mL) was heated at reflux in a Dean-Stark for 6 hours. It was allowed to cool to room temperature, the solid obtained was filtered and the filtrate was washed with saturated solution of NaHCO₃. The phases were separated and the aqueous phase was extracted with AcOEt. The combined organic layers were dried over Na₂SO₄ and then concentrated to dryness. The crude product obtained was purified by chromatography on silica gel using hexane/AcOEt mixtures of increasing polarity as eluent, to afford 11.2 g of the title compound (yield: 76%).

### (b) Title Compound

To a solution of the compound obtained above (3.17 g, 13.09 mmol) and tert-butyl [(3R)-pyrrolidin-3-yl]carbamate (2.2 g, 11.9 mmol) in EtOH (40 mL) under argon atmosphere, DIEA was added (3.4 mL, 19.5 mmol) and the resulting mixture was stirred at reflux for 6 hours. It was allowed to cool to room temperature and then concentrated to dryness. The crude product obtained was purified by chromatography on silica gel using hexane/AcOEt mixtures of increasing polarity as eluent, to afford 4.33 g of the title compound (yield: 100%)

LC-MS (Method 1): t_{R}=10.47 min; m/z=392 (MH⁺).

### Reference Examples 20-22

Following a similar procedure to that described in reference example 19, but using appropriate starting materials instead of tert-butyl[(3R)-pyrrolidin-3-yl]carbamate, the following compounds were obtained:

| Reference | | | Method | | m/z |
|---|---|---|---|---|---|
| example | Name | Starting material | (LC-MS) | t_{R} (min) | (MH⁺) |
| 20 | tert-Butyl 4-[6-chloro-2-(2,5- | 1-(tert- | 1 | 10.50 | 406 |
| | dimethylpyrrol-1- | Butoxycarbonyl)homopiperazine | | | |
| | yl)pyrimidin-4-yl]- | | | | |
| | [1,4]diazepane-1- | | | | |
| | carboxylate | | | | |
| 21 | 4-Chloro-2-(2,5- | 1-methylpiperazine | 1 | 8.65 | 306 |
| | dimethylpyrrol-1-yl)-6-(4- | | | | |
| | methylpiperazin-1- | | | | |
| | yl)pyrimidine | | | | |
| 22 | 1-[6-Chloro-2-(2,5-dimethyl- | 1-methylhomopiperazine | 1 | 8.66 | 320 |
| | pyrrol-1-yl)pyrimidin-4-yl]-4- | | | | |
| | methyl-[1,4]diazepane | | | | |

### Reference Example 23

### tert-Butyl {(3R)-1-[2-(2,5-dimethyl-1H-pyrrol-1-yl)-6-chloropyrimidin-4-yl]pyrrolidin-3-yl}methylcarbamate

To a suspension of 55% NaH (480 mg, 10 mmol) in DMF (12 mL), the compound obtained in reference example 19 (2 g, 6.27 mmol) was added and the resulting mixture was stirred at room temperature for 45 min. Then, MeI (1.17 mL, 18.8 mmol) was added and it was stirred at room temperature for 18 hours. Some drops of water were added, the solvents were evaporated to dryness and the residue was partitioned between AcOEt and 0.2M solution of NaHCO₃. The organic phase was separated and dried over Na₂SO₄ and then concentrated to dryness. The crude product obtained was purified by chromatography on silica gel using hexane/AcOEt mixtures of increasing polarity as eluent, to afford 1.26 g of the title compound (yield: 52%).

LC-MS (Method 1): t_{R}=10.87 min; m/z=406 (MH⁺).

### Reference Example 24

### tert-Butyl {(3R)-1-[2-(2,5-dimethyl-1H-pyrrol-1-yl)-6-chloropyrimidin-4-yl]pyrrolidin-3-yl}ethylcarbamate

Following a similar procedure to that described in reference example 23, but using EtI instead of MeI, the desired compound was obtained (yield: 61%).

LC-MS (Method 1): t_{R}=11.39 min; m/z=420 (MH⁺).

### Reference Example 25

### 2-Amino-6-chloro-4-phenylaminopyrimidine

To a solution of 2-amino-4,6-dichloropyrimidine (6 g, 26.8 mmol) and DIEA (5.1 mL, 29.2 mmol) in dioxane (32 mL) under argon atmosphere, aniline was added (2.45 g, 26.8 mmol) and the resulting mixture was stirred at reflux for 18 hours. The solvent was evaporated and the residue was partitioned between AcOEt and 0.2M solution of NaHCO₃. The phases were separated and the organic phase was dried over Na₂SO₄ and then concentrated to dryness, to afford 4.3 g of the title compound (yield: 79%).

LC-MS (Method 1): t_{R}=5.98 min; m/z=221 (MH⁺).

### Example 1

### 2-Amino-4-phenylamino-6-(4-methyl-[1,4]diazepan-1-yl)pyrimidine

A mixture of the compound obtained in reference example 1 (150 mg, 0.62 mmol), in a dioxane/HCl_{(g)} solution (3 mL) was stirred 15 min at room temperature. It was concentrated to dryness and the resulting residue was suspended in EtOH (4 mL). Aniline (0.085 mL, 0.93 mmol) was added and the mixture was stirred at reflux overnight. The mixture was allowed to cool, the solvent was evaporated and the residue was partitioned between AcOEt and saturated solution of NaHCO₃. The phases were separated and the organic phase was dried over Na₂SO₄ and then concentrated to dryness. The crude product obtained was purified by chromatography on silica gel using CHCl₃/MeOH mixtures of increasing polarity as eluent, to afford 108 mg of the title compound (yield: 29%).

LC-MS (Method 1): t_{R}=4.80 min; m/z=299 (MH⁺).

### Example 2

### 2-Amino-4-phenylamino-6-(4-methylpiperazin-1-yl)pyrimidine

Following a similar procedure to that described in example 1, but using the compound obtained in reference example 2, the desired compound was obtained (yield: 46%).

LC-MS (Method 1): t_{R}=6.03 min; m/z=285 (MH⁺).

### Example 3

### 2-Amino-4-benzylamino-6-(4-methyl-[1,4]diazepan-1-yl)pyrimidine

A mixture of the compound obtained in reference example 1 (150 mg, 0.60 mmol) in benzylamine (0.5 mL) was irradiated in a multimode microwave at 170° C. for 40 min. It was concentrated to dryness and the crude product obtained was purified by chromatography on silica gel using AcOEt/MeOH mixtures of increasing polarity, to afford 140 mg of the title compound (yield: 74%).

LC-MS (Method 1): t_{R}=4.77 min; m/z=313 (MH⁺).

### Examples 4-6

Following a similar procedure to that described in example 3, but using the corresponding starting materials in each case, the following compounds were obtained:

| | | | Method | t_{R} | m/z |
|---|---|---|---|---|---|
| Example | Name | Starting materials | (LC-MS) | (min) | (MH⁺) |
| 4 | 2-Amino-4-benzylamino-6-(4- | Reference example | 1 | 5.24 | 299 |
| | methylpiperazin-1-yl)pyrimidine | 2 and | | | |
| | | benzylamine | | | |
| 5 | 2-Amino-6-(4-methyl- | Reference example | 1 | 5.48 | 327 |
| | [1,4]diazepan-1-yl)-4-((1R)-1- | 1 and | | | |
| | phenylethylamino)pyrimidine | (R)-(+)-α- | | | |
| | | methylbenzylamine | | | |
| 6 | 2-Amino-6-(4-methyl- | Reference example | 1 | 5.46 | 327 |
| | [1,4]diazepan-1-yl)-4-((1S)-1- | 1 and | | | |
| | phenylethylamino)pyrimidine | (S)-(−)-α- | | | |
| | | methylbenzylamine | | | |

### Example 7

### 2-Amino-4-(4-chlorophenylamino)-6-(4-methyl-[1,4]diazepan-1-yl)pyrimidine

A mixture of the compound obtained in reference example 1 (70 mg, 0.28 mmol) in a dioxane/HCl_{(g)} solution (1.5 mL) was stirred 15 min at room temperature. It was concentrated to dryness and the resulting residue was suspended in EtOH (4 mL). 4-Chloroaniline (138 mg, 0.84 mmol) was added and the mixture was irradiated in a multimode microwave at 125° C. for 40 min. The solvent was evaporated and the residue was dissolved in AcOEt and was washed twice with a 0.5N NaOH solution. The organic phase was dried over anhydrous Na₂SO₄ and was concentrated to dryness. The crude product obtained was purified by chromatography on silica gel using as eluent CHCl₃/MeOH mixtures of increasing polarity, to afford 32 mg of the title compound (yield: 34%).

LC-MS (Method 1): t_{R}=6.02 min; m/z=333 (MH⁺).

### Examples 8-112

Following a similar procedure to that described in example 7, but using the corresponding starting materials in each case, the following compounds were obtained:

| | | | Method | t_{R} | m/z |
|---|---|---|---|---|---|
| Example | Name | Starting materials | (LC-MS) | (min) | (MH⁺) |
| 8 | 2-amino-4-(4- | Reference example 1 | 1 | 5.60 | 313 |
| | methylphenylamino)-6-(4- | and | | | |
| | methyl-[1,4]diazepan-1- | p-toluidine | | | |
| | yl)pyrimidine | | | | |
| 9 | 2-amino-4-(3- | Reference example 1 | 1 | 5.60 | 313 |
| | methylphenylamino)-6-(4- | and | | | |
| | methyl-[1,4]diazepan-1- | m-toluidine | | | |
| | yl)pyrimidine | | | | |
| 10 | 2-amino-4-(2- | Reference example 1 | 1 | 5.30 | 313 |
| | methylphenylamino)-6-(4- | and | | | |
| | methyl-[1,4]diazepan-1- | o-toluidine | | | |
| | yl)pyrimidine | | | | |
| 11 | 2-amino-4-(2,4- | Reference example 1 | 1 | 5.86 | 327 |
| | dimethylphenylamino)-6-(4- | and | | | |
| | methyl-[1,4]diazepan-1- | 2,4-dimethylaniline | | | |
| | yl)pyrimidine | | | | |
| 12 | 2-amino-4-(2- | Reference example 1 | 1 | 4.75 | 315 |
| | hydroxyphenylamino)-6-(4- | and | | | |
| | methyl-[1,4]diazepan-1- | 2-aminophenol | | | |
| | yl)pyrimidine | | | | |
| 13 | 2-amino-4-(3- | Reference example 1 | 1 | 6.22 | 333 |
| | chlorophenylamino)-6-(4- | and | | | |
| | methyl-[1,4]diazepan-1- | 3-chloroaniline | | | |
| | yl)pyrimidine | | | | |
| 14 | 2-amino-6-(4-methyl- | Reference example 1 | 1 | 5.11 | 329 |
| | [1,4]diazepan-1-yl)-4-(4- | and | | | |
| | methoxyphenylamino)pyrimidine | p-anisidine | | | |
| 15 | 2-amino-6-(4-methyl- | Reference example 1 | 1 | 5.32 | 329 |
| | [1,4]diazepan-1-yl)4-(3- | and | | | |
| | methoxyphenylamino)pyrimidine | m-anisidine | | | |
| 16 | 2-amino-4-(4-fluoro-2- | Reference example 1 | 1 | 5.70 | 331 |
| | methylphenylamino)-6-(4- | and | | | |
| | methyl-[1,4]diazepan-1- | 4-fluoro-2-methylaniline | | | |
| | yl)pyrimidine | | | | |
| 17 | 2-amino-4-(3- | Reference example 1 | 1 | 6.17 | 379 |
| | bromophenylamino)-6-(4- | and | | | |
| | methyl-[1,4]diazepan-1- | 3-bromoaniline | | | |
| | yl)pyrimidine | | | | |
| 18 | 2-amino-4-(3- | Reference example 1 | 1 | 5.43 | 317 |
| | fluorophenylamino)-6-(4- | and | | | |
| | methyl-[1,4]diazepan-1- | 3-fluoroaniline | | | |
| | yl)pyrimidine | | | | |
| 19 | 2-amino-4-(4- | Reference example 1 | 1 | 5.32 | 317 |
| | fluorophenylamino)-6-(4- | and | | | |
| | methyl-[1,4]diazepan-1- | 4-fluoroaniline | | | |
| | yl)pyrimidine | | | | |
| 20 | 2-amino-4-(1H-indol-6-ilamino)- | Reference example 1 | 1 | 5.26 | 338 |
| | 6-(4-methyl-[1,4]diazepan-1- | and | | | |
| | yl)pyrimidine | 6-aminoindol | | | |
| 21 | 2-amino-4-(benzo[1,3]dioxol-5- | Reference example 1 | 1 | 4.83 | 343 |
| | ylamino)-6-(4-methyl- | and | | | |
| | [1,4]diazepan-1-yl)pyrimidine | 3,4- | | | |
| | | methylendioxyaniline | | | |
| 22 | 2-amino-4-(3,4- | Reference example 1 | 1 | 7.07 | 367 |
| | dichlorophenylamino)-6-(4- | and | | | |
| | methyl-[1,4]diazepan-1- | 3,4-dichloroaniline | | | |
| | yl)pyrimidine | | | | |
| 23 | 2-amino-4-(benzo[b]thiophen- | Reference example 1 | 1 | 6.13 | 355 |
| | 5-ylamino)-6-(4-methyl- | and | | | |
| | [1,4]diazepan-1-yl)pyrimidine | 5-aminobenzothiophene | | | |
| 24 | 2-amino-4-(3- | Reference example 1 | 1 | 5.87 | 345 |
| | (methylthio)phenylamino)-6-(4- | and | | | |
| | methyl-[1,4]diazepan-1- | 3-(methylthio)aniline | | | |
| | yl)pyrimidine | | | | |
| 25 | 2-amino-6-(4-methyl- | Reference example 1 | 1 | 5.38 | 335 |
| | [1,4]diazepan-1-yl)-4-(2,4- | and | | | |
| | difluorophenylamino)pyrimidine | 2,4-difluoroaniline | | | |
| 26 | 2-amino-6-(4-methyl- | Reference example 1 | 1 | 6.94 | 383 |
| | [1,4]diazepan-1-yl)-4-(4- | and | | | |
| | trifluoromethoxyphenylamino)pyrimidine | 4- | | | |
| | | trifluoromethoxyaniline | | | |
| 27 | 2-amino-4-(biphenyl-3- | Reference example 1 | 1 | 7.17 | 375 |
| | ylamino)-6-(4-methyl- | and | | | |
| | [1,4]diazepan-1-yl)pyrimidine | biphenyl-3-ylamine | | | |
| 28 | 2-amino-4-(1H-indol-7- | Reference example 1 | 1 | 5.51 | 338 |
| | ylamino)-6-(4-methyl- | and | | | |
| | [1,4]diazepan-1-yl)pyrimidine | 7-aminoindol | | | |
| 29 | 2-amino-4-(indan-5-ylamino)-6- | Reference example 1 | 1 | 6.31 | 339 |
| | (4-methyl-[1,4]diazepan-1- | and | | | |
| | yl)pyrimidine | 5-aminoindane | | | |
| 30 | 2-amino-4-(4- | Reference example 1 | 1 | 3.77 | 315 |
| | hydroxyphenylamino)-6-(4- | and | | | |
| | methyl-[1,4]diazepan-1- | 4-aminophenol | | | |
| | yl)pyrimidine | | | | |
| 31 | 2-amino-4-(1H-indazol-5- | Reference example 1 | 1 | 3.76 | 339 |
| | ylamino)-6-(4-methyl- | and | | | |
| | [1,4]diazepan-1-yl)pyrimidine | 5-aminoindazol | | | |
| 32 | 2-amino-4-(1H-indol-5- | Reference example 1 | 1 | 4.72 | 338 |
| | ylamino)-6-(4-methyl- | and | | | |
| | [1,4]diazepan-1-yl)pyrimidine | 5-aminoindol | | | |
| 33 | 2-amino-6-(4-methyl- | Reference example 1 | 1 | 5.35 | 343 |
| | [1,4]diazepan-1-yl)-4-(2-methyl- | and | | | |
| | 4- | 4-methoxy-2- | | | |
| | methoxyphenylamino)pyrimidine | methylaniline | | | |
| 34 | 4-(3-acetylphenylamino)-2- | Reference example 1 | 1 | 4.94 | 341 |
| | amino-6-(4-methyl- | and | | | |
| | [1,4]diazepan-1-yl)pyrimidine | 3-aminoacetophenone | | | |
| 35 | 2-amino-6-(4-methyl- | Reference example 1 | 1 | 6.48 | 349 |
| | [1,4]diazepan-1-yl)-4- | and | | | |
| | (naphtalen-2- | 2-naphthylamine | | | |
| | ylamino)pyrimidine | | | | |
| 36 | 2-amino-6-(4-methyl- | Reference example 1 | 1 | 8.20 | 435 |
| | [1,4]diazepan-1-yl)-4-[3,5- | and | | | |
| | bis(trifluoromethyl)phenylamino]pyrimidine | 3,5- | | | |
| | | bis(trifluoromethyl)aniline | | | |
| 37 | 2-amino-4-(3- | Reference example 1 | 1 | 4.15 | 315 |
| | hydroxyphenylamino)-6-(4- | and | | | |
| | methyl-[1,4]diazepan-1- | 3-aminophenol | | | |
| | yl)pyrimidine | | | | |
| 38 | 2-amino-4-(3,5- | Reference example 1 | 1 | 7.41 | 367 |
| | dichlorophenylamino)-6-(4- | and | | | |
| | methyl-[1,4]diazepan-1-yl- | 3,5-dichloroaniline | | | |
| | pyrimidine | | | | |
| 39 | 2-amino-4-(3- | Reference example 1 | 1 | 4.11 | 356 |
| | acetylaminophenylamino)-6-(4- | and | | | |
| | methyl-[1,4]diazepan-1- | 3-aminoacetanilide | | | |
| | yl)pyrimidine | | | | |
| 40 | 2-amino-4-(3- | Reference example 1 | 1 | 5.26 | 324 |
| | cyanophenylamino)-6-(4- | and | | | |
| | methyl-[1,4]diazepan-1- | 3-cyanoaniline | | | |
| | yl)pyrimidine | | | | |
| 41 | 2-amino-4-(3- | Reference example 1 | 1 | 4.11 | 329 |
| | hydroxymethylphenylamino)-6- | and | | | |
| | (4-methyl-[1,4]diazepan-1- | 3-Aminobenzylic alcohol | | | |
| | yl)pyrimidine | | | | |
| 42 | 2-Amino-4-(2- | Reference example 1 | 1 | 5.31 | 317 |
| | fluorophenylamino)-6-(4- | and | | | |
| | methyl-[1,4]diazepan-1- | 2-fluoroaniline | | | |
| | yl)pyrimidine | | | | |
| 43 | 2-Amino-6-(4-methyl- | Reference example 1 | 1 | 6.91 | 383 |
| | [1,4]diazepan-1-yl)-4-(3- | and | | | |
| | (trifluoromethoxy)phenylamino)pyrimidine | 3- | | | |
| | | (trifluoromethoxy)aniline | | | |
| 44 | 2-Amino-6-([1,4]diazepan-1-yl)- | Reference example 4 | 1 | 4.32 | 285 |
| | 4-(phenylamino)pyrimidine | and | | | |
| | hydrochloride | aniline | | | |
| 45 | 2-Amino-6-([1,4]diazepan-1-yl)- | Reference example 4 | 1 | 4.81 | 303 |
| | 4-(3- | and | | | |
| | fluorophenylamino)pyrimidine | 3-fluoroaniline | | | |
| 46 | 2-Amino-4-(3- | Reference example 4 | 1 | 5.36 | 319 |
| | chlorophenylamino)-6- | and | | | |
| | ([1,4]diazepan-1-yl)pyrimidine | 3-chloroaniline | | | |
| 47 | 2-Amino-6-([1,4]diazepan-1-yl)- | Reference example 4 | 1 | 4.9 | 299 |
| | 4-(3-tolylamino)pyrimidine | and | | | |
| | | 3-methylaniline | | | |
| 48 | 2-Amino-6-([1,4]diazepan-1-yl)- | Reference example 4 | 1 | 4.5 | 299 |
| | 4-(2-tolylamino)pyrimidine | and | | | |
| | | 2-methylaniline | | | |
| 49 | 2-Amino-6-([1,4]diazepan-1-yl)- | Reference example 4 | 1 | 3.36 | 301 |
| | 4-(3- | and | | | |
| | hydroxyphenylamino)pyrimidine | 3-aminophenol | | | |
| 50 | 2-Amino-4-(3-chloro-4- | Reference example 4 | 1 | 5.51 | 337 |
| | fluorophenylamino)-6- | and | | | |
| | ([1,4]diazepan-1-yl)pyrimidine | 3-chloro-4-fluoroaniline | | | |
| 51 | 2-Amino-6-([1,4]diazepan-1-yl)- | Reference example 4 | 1 | 4.58 | 303 |
| | 4-(4- | and | | | |
| | fluorophenylamino)pyrimidine | 4-fluoroaniline | | | |
| 52 | 2-Amino-6-([1,4]diazepan-1-yl)- | Reference example 4 | 1 | 4.58 | 315 |
| | 4-(3- | and | | | |
| | methoxyphenylamino)pyrimidine | 3-methoxyaniline | | | |
| 53 | 2-Amino-6-([1,4]diazepan-1-yl)- | Reference example 4 | 1 | 6.47 | 353 |
| | 4-(3,5- | and | | | |
| | dichlorophenylamino)pyrimidine | 3,5-dichloroaniline | | | |
| 54 | 2-Amino-6-([1,4]diazepan-1-yl)- | Reference example 4 | 1 | 5.06 | 321 |
| | 4-(3,4- | and | | | |
| | difluorophenylamino)pyrimidine | 3,4-difluoroaniline | | | |
| 55 | 2-Amino-6-([1,4]diazepan-1-yl)- | Reference example 4 | 1 | 5.06 | 317 |
| | 4-(4-fluoro-3- | and | | | |
| | methylphenylamino)pyrimidine | 4-fluoro-3-methylaniline | | | |
| 56 | 2-Amino-6-([1,4]diazepan-1-yl)- | Reference example 20 | 1 | 5.37 | 339 |
| | 4-(2,3,4- | and | | | |
| | trifluorophenylamino)pyrimidine | 2,3,4-trifluoroaniline | | | |
| 57 | 2-Amino-6-([1,4]diazepan-1-yl)- | Reference example 20 | 1 | 5.93 | 339 |
| | 4-(3,4,5- | and | | | |
| | trifluorophenylamino)pyrimidine | 3,4,5-trifluoroaniline | | | |
| 58 | 2-Amino-4-(5-chloro-2- | Reference example 20 | 1 | 5.54 | 337 |
| | fluorophenylamino)-6- | and | | | |
| | ([1,4]diazepan-1-yl)pyrimidine | 5-chloro-2-fluoroaniline | | | |
| 59 | 2-Amino-6-([1,4]diazepan-1-yl)- | Reference example 20 | 1 | 5.03 | 321 |
| | 4-(2,5- | and | | | |
| | difluorophenylamino)pyrimidine | 2,5-difluoroaniline | | | |
| 60 | 2-Amino-4-(2- | Reference example 21 | 1 | 5.83 | 319 |
| | chlorophenylamino)-6-(4- | and | | | |
| | methylpiperazin-1-yl)pyrimidine | 2-chloroaniline | | | |
| 61 | 2-Amino-6-(4-methylpiperazin- | Reference example 21 | 1 | 5.94 | 335 |
| | 1-yl)-4-(1- | and | | | |
| | naphthylamino)pyrimidine | 1-naphthylamine | | | |
| 62 | 2-Amino-6-(4-methyl- | Reference example 1 | 1 | 5.78 | 331 |
| | [1,4]diazepan-1-yl)-4-(3-fluoro- | and | | | |
| | 2- | 3-fluoro-2-methylaniline | | | |
| | methylphenylamino)pyrimidine | | | | |
| 63 | 2-Amino-4-(3,4- | Reference example 1 | 1 | 5.86 | 335 |
| | difluorophenylamino)-6-(4- | and | | | |
| | methyl-[1,4]diazepan-1- | 3,4-difluoroaniline | | | |
| | yl)pyrimidine | | | | |
| 64 | 2-Amino-4-(3-chloro-4- | Reference example 1 | 1 | 6.27 | 351 |
| | fluorophenylamino)-6-(4- | and | | | |
| | methyl-[1,4]diazepan-1- | 3-chloro-4-fluoro aniline | | | |
| | yl)pyrimidine | | | | |
| 65 | 2-Amino-6-(4-methyl- | Reference example 1 | 1 | 6.83 | 353 |
| | [1,4]diazepan-1-yl)-4-(3,4,5- | and | | | |
| | trifluorophenylamino)pyrimidine | 3,4,5-trifluoroaniline | | | |
| 66 | 2-Amino-4-(2-fluoro-3- | Reference example 22 | 1 | 6.98 | 385 |
| | (trifluoromethyl)phenylamino)- | and | | | |
| | 6-(4-methyl-[1,4]diazepan-1- | 2-fluoro-3- | | | |
| | yl)pyrimidine | trifluoromethylaniline | | | |
| 67 | 2-Amino-4-(5-fluoro-2- | Reference example 22 | 1 | 6.05 | 331 |
| | methylphenylamino)-6-(4- | and | | | |
| | methyl-[1,4]diazepan-1- | 5-fluoro-2-methylaniline | | | |
| | yl)pyrimidine | | | | |
| 68 | 2-Amino-4-(2,5- | Reference example 22 | 1 | 6.13 | 335 |
| | difluorophenylamino)-6-(4- | and | | | |
| | methyl-[1,4]diazepan-1- | 2,5-difluoroaniline | | | |
| | yl)pyrimidine | | | | |
| 69 | 2-Amino-6-(4-methyl- | Reference example 22 | 1 | 6.33 | 353 |
| | [1,4]diazepan-1-yl)-4-(2,4,5- | and | | | |
| | trifluorophenylamino)pyrimidine | 2,4,5-trifluoroaniline | | | |
| 70 | 2-Amino-6-(4-methyl- | Reference example 22 | 1 | 6.13 | 353 |
| | [1,4]diazepan-1-yl)-4-(2,3,4- | and | | | |
| | trifluorophenylamino)pyrimidine | 2,3,4-trifluoroaniline | | | |
| 71 | 2-Amino-4-(4- | Reference example 3 | 1 | 4.53 | 289 |
| | fluorophenylamino)-6- | and | | | |
| | (piperazin-1-yl)pyrimidine | 4-fluoroaniline | | | |
| 72 | 2-Amino-4-(3- | Reference example 3 | 1 | 4.74 | 289 |
| | fluorophenylamino)-6- | and | | | |
| | (piperazin-1-yl)pyrimidine | 3-fluoroaniline | | | |
| 73 | 2-Amino-4-(3- | Reference example 3 | 1 | 5.51 | 305 |
| | chlorophenylamino)-6- | and | | | |
| | (piperazin-1-yl)pyrimidine | 3-chloroaniline | | | |
| 74 | 2-Amino-4-(2,3- | Reference example 22 | 1 | 5.62 | 335 |
| | difluorophenylamino)-6-(4- | and | | | |
| | methyl-[1,4]diazepan-1- | 2,3-difluoroaniline | | | |
| | yl)pyrimidine | | | | |
| 75 | 2-Amino-4-(4- | Reference example 2 | 1 | 5.51 | 303 |
| | fluorophenylamino)-6-(4- | and | | | |
| | methylpiperazin-1-yl)pyrimidine | 4-fluoroaniline | | | |
| 76 | 2-Amino-4-(3- | Reference example 2 | 1 | 5.73 | 303 |
| | fluorophenylamino)-6-(4- | and | | | |
| | methylpiperazin-1-yl)pyrimidine | 3-fluoroaniline | | | |
| 77 | 2-Amino-4-(3- | Reference example 2 | 1 | 6.28 | 319 |
| | chlorophenylamino)-6-(4- | and | | | |
| | methylpiperazin-1-yl)pyrimidine | 3-chloroaniline | | | |
| 78 | 2-Amino-4-(2,4- | Reference example 11 | 1 | 5.08 | 307 |
| | difluorophenylamino)-6-(3- | and | | | |
| | (methylamino)azetidin-1- | 2,4-difluoroaniline | | | |
| | yl)pyrimidine | | | | |
| 79 | 2-Amino-6-(3- | Reference example 11 | 1 | 6.26 | 339 |
| | (methylamino)azetidin-1-yl)-4- | and | | | |
| | (3- | 3-trifluoromethylaniline | | | |
| | (trifluoromethyl)phenylamino)pyrimidine | | | | |
| 80 | 2-Amino-4-(2- | Reference example 11 | 1 | 4.88 | 289 |
| | fluorophenylamino)-6-(3- | and | | | |
| | (methylamino)azetidin-1- | 2-fluoroaniline | | | |
| | yl)pyrimidine | | | | |
| 81 | 2-Amino-4-(4-fluoro-3- | Reference example 8 | 1 | 5.65 | 317 |
| | methylphenylamino)-6-((3R)-3- | and | | | |
| | (methylamino)pyrrolidin-1- | 4-fluoro-3-methylaniline | | | |
| | yl)pyrimidine | | | | |
| 82 | 2-Amino-4-(3- | Reference example 8 | 1 | 6.04 | 313 |
| | ethylphenylamino)-6-((3R)-3- | and | | | |
| | (methylamino)pyrrolidin-1- | 3-ethylaniline | | | |
| | yl)pyrimidine | | | | |
| 83 | 2-Amino-6-((3R)-3- | Reference example 8 | 1 | 6.22 | 339 |
| | (methylamino)pyrrolidin-1-yl)-4- | and | | | |
| | (3,4,5- | 3,4,5-trifluoroaniline | | | |
| | trifluorophenylamino)pyrimidine | | | | |
| 84 | 6-(3-(Methylamino)azetidin-1- | Reference example 11 | 1 | 4.79 | 303 |
| | yl)-N⁴-(3,4,5- | and | | | |
| | trifluorophenyl)pyrimidine-2,4- | 3,4,5-trifluoroaniline | | | |
| | diamine | | | | |
| 85 | N⁴-(3-Chloro-4-fluorophenyl)-6- | Reference example 18 | 1 | 5.96 | 337 |
| | [(3S)-3- | and | | | |
| | (methylamino)pyrrolidin-1- | 3-chloro-4-fluoroaniline | | | |
| | yl]pyrimidine-2,4-diamine | | | | |
| 86 | N⁴-(3-Chlorophenyl)-6- | Reference example 16 | 1 | 6.22 | 345 |
| | (octahydropyrrolo[3,4-b]pyridin- | and | | | |
| | 6-yl)pyrimidine-2,4-diamine | 3-chloroaniline | | | |
| 87 | N⁴-(3-Chloro-4-fluorophenyl)-6- | Reference example 16 | 1 | 6.40 | 363 |
| | (octahydropyrrolo[3,4-b]pyridin- | and | | | |
| | 6-yl)pyrimidine-2,4-diamine | 3-chloro-4-fluoroaniline | | | |
| 88 | N⁴-(3-Methylphenyl)-6- | Reference example 16 | 1 | 5.89 | 325 |
| | (octahydropyrrolo[3,4-b]pyridin- | and | | | |
| | 6-yl)pyrimidine-2,4-diamine | m-toluidine | | | |
| 89 | N⁴-(4-Fluoro-3-methylphenyl)- | Reference example 16 | 1 | 6.09 | 343 |
| | 6-(octahydropyrrolo[3,4- | and | | | |
| | b]pyridin-6-yl)pyrimidine-2,4- | 4-fluoro-3-methylaniline | | | |
| | diamine | | | | |
| 90 | 6-[(3S)-3- | Reference example 18 | 1 | 5.46 | 299 |
| | (methylamino)pyrrolidin-1-yl]- | and | | | |
| | N⁴-m-tolylpyrimidine-2,4- | m-toluidine | | | |
| | diamine | | | | |
| 91 | N⁴-(3,4-Difluorophenyl)-6-[(3S)- | Reference example 18 | 2 | 2.23 | 321 |
| | 3-(methylamino)pyrrolidin-1- | and | | | |
| | yl]pyrimidine-2,4-diamine | 3,4-difluoroaniline | | | |
| 92 | N⁴-(3-Trifluoromethylphenyl)-6- | Reference example 18 | 2 | 2.39 | 353 |
| | [(3S)-3- | and | | | |
| | (methylamino)pyrrolidin-1- | 3-trifluoromethylaniline | | | |
| | yl]pyrimidine-2,4-diamine | | | | |
| 93 | 3-[2-Amino-6-[(3R)-3- | Reference example 8 | 2 | 2.03 | 315 |
| | (methylamino)pyrrolidin-1- | and | | | |
| | yl]pyrimidin-4-ylamino]-2- | 3-amino-2-methylphenol | | | |
| | methylphenol | | | | |
| 94 | N⁴-(4-Fluoro-3- | Reference example 8 | 2 | 2.14 | 333 |
| | methoxyphenyl)-6-[(3R)-3- | and | | | |
| | (methylamino)pyrrolidin-1- | 4-fluoro-3- | | | |
| | yl]pyrimidine-2,4-diamine | methoxyaniline | | | |
| 95 | N⁴-(2,4-Difluoro-3- | Reference example 8 | 2 | 2.25 | 351 |
| | methoxyphenyl)-6-[(3R)-3- | and | | | |
| | (methylamino)pyrrolidin-1- | 2,4-difluoro-3- | | | |
| | yl]pyrimidine-2,4-diamine | methoxyaniline | | | |
| 96 | N⁴-(2-Fluorophenyl)-6-[(3S)-3- | Reference example 18 | 1 | 4.95 | 303 |
| | (methylamino)pyrrolidin-1- | and | | | |
| | yl]pyrimidine-2,4-diamine | 2-fluoroaniline | | | |
| 97 | N⁴-(3-Fluorophenyl)-6-[(3S)-3- | Reference example 18 | 1 | 5.25 | 303 |
| | (methylamino)pyrrolidin-1- | and | | | |
| | yl]pyrimidine-2,4-diamine | 3-fluoroaniline | | | |
| 98 | 6-[(3R)-3-aminopyrrolidin-1-yl]- | Reference example 10 | 1 | 5.17 | 285 |
| | N⁴-m-tolylpyrimidine-2,4- | and | | | |
| | diamine | m-toluidine | | | |
| 99 | 6-[(3R)-3-aminopyrrolidin-1-yl]- | Reference example 10 | 2 | 2.22 | 323 |
| | N⁴-(3-chloro-4- | and | | | |
| | fluorophenyl)pyrimidine-2,4- | 3-chloro-4-fluoroaniline | | | |
| | diamine | | | | |
| 100 | 6-[(3R)-3-aminopyrrolidin-1-yl]- | Reference example 10 | 2 | 2.01 | 289 |
| | N⁴-(2-fluorophenyl)pyrimidine- | and | | | |
| | 2,4-diamine | 2-fluoroaniline | | | |
| 101 | 6-[(3R)-3-aminopyrrolidin-1-yl]- | Reference example 10 | 1 | 5.39 | 303 |
| | N⁴-(4-fluoro-3- | and | | | |
| | methylphenyl)pyrimidine-2,4- | 4-fluoro-3-methylaniline | | | |
| | diamine | | | | |
| 102 | 6-[(3R)-3-aminopyrrolidin-1-yl]- | Reference example 10 | 1 | 5.30 | 307 |
| | N⁴-(3,4- | and | | | |
| | difluorophenyl)pyrimidine-2,4- | 3,4-difluoroaniline | | | |
| | diamine | | | | |
| 103 | 6-[(3R)-3-aminopyrrolidin-1-yl]- | Reference example 10 | 1 | 5.04 | 289 |
| | N⁴-(3-fluorophenyl)pyrimidine- | and | | | |
| | 2,4-diamine | 3-fluoroaniline | | | |
| 104 | 3-[2-Amino-6-(3- | Reference example 11 | 1 | 3.98 | 287 |
| | (methylamino)azetidin-1-yl)- | and | | | |
| | pyrimidin-4-ylamino]phenol | 3-aminophenol | | | |
| 105 | N⁴-(3-Methoxyphenyl)-6-(3- | Reference example 11 | 1 | 5.03 | 301 |
| | (methylamino)azetidin-1-yl)- | and | | | |
| | pyrimidine-2,4-diamine | 3-methoxyaniline | | | |
| 106 | 6-(3-(Methylamino)azetidin-1- | Reference example 11 | 1 | 6.16 | 321 |
| | yl)-N⁴-naphthalen-2- | and | | | |
| | ylpyrimidine-2,4-diamine | naphthalen-2-ylamine | | | |
| 107 | 3-[2-Amino-6-(3- | Reference example 11 | 1 | 4.98 | 296 |
| | (methylamino)azetidin-1-yl)- | and | | | |
| | pyrimidin-4- | 3-aminobenzonitrile | | | |
| | ylamino]benzonitrile | | | | |
| 108 | N⁴-(4-Fluoro-3- | Reference example 11 | 1 | 5.23 | 319 |
| | methoxyphenyl)-6-(3- | and | | | |
| | (methylamino)azetidin-1-yl)- | 4-fluoro-3- | | | |
| | pyrimidine-2,4-diamine | methoxyaniline | | | |
| 109 | 5-[2-Amino-6-(3- | Reference example 11 | 1 | 5.34 | 314 |
| | (methylamino)azetidin-1-yl)- | and | | | |
| | pyrimidin-4-ylamino]-2-fluoro- | 5-amino-2- | | | |
| | benzonitrile | fluorobenzonitrile | | | |
| 110 | N⁴-(3-Ethylphenyl)-6-(3- | Reference example 11 | 1 | 6.03 | 299 |
| | (methylamino)azetidin-1-yl)- | and | | | |
| | pyrimidine-2,4-diamine | 4-ethylaniline | | | |
| 111 | N⁴-(2,4-Difluoro-3- | Reference example 11 | 1 | 5.71 | 337 |
| | methoxyphenyl)-6-(3- | and | | | |
| | (methylamino)azetidin-1-yl)- | 2,4-difluoro-3- | | | |
| | pyrimidine-2,4-diamine | methoxyaniline | | | |
| 112* | N⁴-(2,3-Difluorophenyl)-6-(3- | Reference example 11 | 1 | 4.30 | 307 |
| | (methylamino)azetidin-1-yl)- | and | | | |
| | pyrimidine-2,4-diamine | 2,3-difluoroaniline | | | |

*The reaction is carried out in BuOH instead of EtOH

### Examples 113-140

Following a similar procedure to that described in example 7, but using the corresponding starting materials in each case and irradiating in a multimode microwave at 140° C. for 50 min, the following compounds were obtained:

| | | | Method | t_{R} | |
|---|---|---|---|---|---|
| Example | Name | Starting materials | (LC-MS) | (min) | m/z |
| 113 | 2-Amino-6-(3- | Reference example | 1 | 5.17 | 285 |
| | (methylamino)azetidin-1-yl)-4- | 11 and | | | |
| | (2-tolylamino)pyrimidine | 2-methylaniline | | | |
| 114 | 2-Amino-4-(3-chloro-2- | Reference example | 1 | 5.84 | 337 |
| | fluorophenylamino)-6-((3R)-3- | 8 and | | | |
| | (methylamino)pyrrolidin-1- | 3-chloro-2- | | | |
| | yl)pyrimidine | fluoroaniline | | | |
| 115 | 2-Amino-4-(2,3- | Reference example | 1 | 5.27 | 321 |
| | difluorophenylamino)-6-((3R)-3- | 8 and | | | |
| | (methylamino)pyrrolidin-1- | 2,3-difluoroaniline | | | |
| | yl)pyrimidine | | | | |
| 116 | 2-Amino-4-(4-fluoro-2- | Reference example | 1 | 5.32 | 317 |
| | methylphenylamino)-6-((3R)-3- | 8 and | | | |
| | (methylamino)pyrrolidin-1- | 4-fluoro-2- | | | |
| | yl)pyrimidine | methylaniline | | | |
| 117 | 2-Amino-4-(3-chloro-2- | Reference example | 1 | 5.88 | 333 |
| | methylphenylamino)-6-((3R)-3- | 8 and | | | |
| | (methylamino)pyrrolidin-1- | 3-chloro-2- | | | |
| | yl)pyrimidine | methylaniline | | | |
| 118 | 2-Amino-4-(2-chloro-4- | Reference example | 1 | 5.58 | 337 |
| | fluorophenylamino)-6-((3R)-3- | 8 and | | | |
| | (methylamino)pyrrolidin-1- | 2-chloro-4- | | | |
| | yl)pyrimidine | fluoroaniline | | | |
| 119 | N⁴-(3-Chloro-2-fluorophenyl)-6- | Reference example | 1 | 4.98 | 323 |
| | (3-(methylamino)azetidin-1- | 11 and | | | |
| | yl)pyrimidine-2,4-diamine | 3-chloro-2- | | | |
| | | fluoroaniline | | | |
| 120 | N⁴-(3-Fluoro-2-methylphenyl)-6- | Reference example | 1 | 5.30 | 303 |
| | (3-(methylamino)azetidin-1- | 11 and | | | |
| | yl)pyrimidine-2,4-diamine | 3-fluoro-2- | | | |
| | | methylaniline | | | |
| 121 | 6-(3-(Methylamino)azetidin-1- | Reference example | 1 | 4.65 | 325 |
| | yl)-N⁴-(2,3,4- | 11 and | | | |
| | trifluorophenyl)pyrimidine-2,4- | 2,3,4-trifluoroaniline | | | |
| | diamine | | | | |
| 122 | N⁴-(4-Fluoro-2-methylphenyl)-6- | Reference example | 1 | 5.20 | 303 |
| | (3-(methylamino)azetidin-1- | 11 and | | | |
| | yl)pyrimidine-2,4-diamine | 4-fluoro-2- | | | |
| | | methylaniline | | | |
| 123 | N⁴-(2-Chloro-4-fluorophenyl)-6- | Reference example | 1 | 4.60 | 323 |
| | (3-(methylamino)azetidin-1- | 11 and | | | |
| | yl)pyrimidine-2,4-diamine | 2-chloro-4- | | | |
| | | fluoroaniline | | | |
| 124 | 6-[(3R)-3- | Reference example | 1 | 5.60 | 339 |
| | (Methylamino)pyrrolidin-1-yl]- | 8 and | | | |
| | N⁴-(2,3,4- | 2,3,4-trifluoroaniline | | | |
| | trifluorophenyl)pyrimidine-2,4- | | | | |
| | diamine | | | | |
| 125 | N⁴-(2,3-Dichlorophenyl)-6- | Reference example | 1 | 6.28 | 353 |
| | [(3R)-3-(methylamino)pyrrolidin- | 8 and | | | |
| | 1-yl]pyrimidine-2,4-diamine | 2,3-dichloroaniline | | | |
| 126 | N⁴-(2,3-Dimethylphenyl)-6- | Reference example | 1 | 5.59 | 313 |
| | [(3R)-3-(methylamino)pyrrolidin- | 8 and | | | |
| | 1-yl]pyrimidine-2,4-diamine | 2,3-dimethylaniline | | | |
| 127 | 6-[(3R)-3- | Reference example | 2 | 2.34 | 313 |
| | (Dimethylamino)pyrrolidin-1-yl]- | 17 and | | | |
| | N⁴-m-tolyl-pyrimidine-2,4- | m-toluidine | | | |
| | diamine | | | | |
| 128 | N⁴-(3-Chloro-4-fluorophenyl)-6- | Reference example | 2 | 2.44 | 351 |
| | [(3R)-3- | 17 and | | | |
| | (dimethylamino)pyrrolidin-1- | 3-chloro-4- | | | |
| | yl]pyrimidine-2,4-diamine | fluoroaniline | | | |
| 129 | N⁴-(3,4-Difluorophenyl)-6-[(3R)- | Reference example | 2 | 2.35 | 335 |
| | 3-(dimethylamino)pyrrolidin-1- | 17 and | | | |
| | yl]pyrimidine-2,4-diamine | 3,4-difluoroaniline | | | |
| 130 | N⁴-(4-Fluoro-3-methylphenyl)-6- | Reference example | 2 | 2.38 | 331 |
| | [(3R)-3- | 17 and | | | |
| | (dimethylamino)pyrrolidin-1- | 4-fluoro-3- | | | |
| | yl]pyrimidine-2,4-diamine | methylaniline | | | |
| 131 | N⁴-(3-Chloro-2-fluorophenyl)-6- | Reference example | 2 | 2.44 | 351 |
| | [(3R)-3- | 17 and | | | |
| | (dimethylamino)pyrrolidin-1- | 3-chloro-2- | | | |
| | yl]pyrimidine-2,4-diamine | fluoroaniline | | | |
| 132 | 6-[(3R)-3-Aminopyrrolidin-1-yl]- | Reference example | 2 | 2.13 | 295 |
| | N⁴-(3-ethynylphenyl)pyrimidine- | 10 and | | | |
| | 2,4-diamine | 3-ethynylaniline | | | |
| 133 | 6-[(3R)-3-Aminopyrrolidin-1-yl]- | Reference example | 2 | 2.26 | 325 |
| | N⁴-(3,4,5- | 10 and | | | |
| | trifluorophenyl)pyrimidine-2,4- | 3,4,5-trifluoroaniline | | | |
| | diamine | | | | |
| 134 | 6-[(3R)-3-Aminopyrrolidin-1-yl]- | Reference example | 2 | 2.10 | 303 |
| | N⁴-(4-fluoro-2- | 10 and | | | |
| | methylphenyl)pyrimidine-2,4- | 4-fluoro-2- | | | |
| | diamine | methylaniline | | | |
| 135 | 6-[(3R)-3-Aminopyrrolidin-1-yl]- | Reference example | 2 | 2.29 | 329 |
| | N⁴-(3- | 10 and | | | |
| | trifluoromethylphenyl)pyrimidine- | 3- | | | |
| | 2,4-diamine | trifluoromethylaniline | | | |
| 136 | 6-[(3R)-3- | Reference example | 1 | 5.68 | 299 |
| | (Dimethylamino)pyrrolidin-1-yl]- | 17 and | | | |
| | N⁴-phenylpyrimidine-2,4- | aniline | | | |
| | diamine | | | | |
| 137 | 6-[(3R)-3- | Reference example | 1 | 5.89 | 317 |
| | (Dimethylamino)pyrrolidin-1-yl]- | 17 and | | | |
| | N⁴-(4-fluorophenyl)pyrimidine- | 4-fluoroaniline | | | |
| | 2,4-diamine | | | | |
| 138 | N⁴-(3-Chlorophenyl)-6-[(3R)-3- | Reference example | 2 | 2.42 | 333 |
| | (dimethylamino)pyrrolidin-1- | 17 and | | | |
| | yl]pyrimidine-2,4-diamine | 3-chloroaniline | | | |
| 139 | 6-[(3R)-3- | Reference example | 2 | 2.24 | 317 |
| | (Dimethylamino)pyrrolidin-1-yl]- | 17 and | | | |
| | N⁴-(2-fluorophenyl)pyrimidine- | 2-fluoroaniline | | | |
| | 2,4-diamine | | | | |
| 140 | 6-[(3R)-3- | Reference example | 2 | 2.30 | 317 |
| | (Dimethylamino)pyrrolidin-1-yl]- | 17 and | | | |
| | N⁴-(3-fluorophenyl)pyrimidine- | 3-fluoroaniline | | | |
| | 2,4-diamine | | | | |

### Example 141

### 2-Amino-6-(4-methyl-[1,4]diazepan-1-yl)-4-(3-trifluoromethylphenylamino)pyrimidine

### Example 142

### 2-Amino-6-([1,4]diazepan-1-yl)-4-(3-trifluoromethylphenylamino)pyrimidine

Following a similar procedure to that described in example 7 but using 3-trifluoromethylaniline instead of 4-chloroaniline, example 141 was obtained (LC-MS (Method 1): t_{R}=6.72 min; m/z=367 (MH⁺)) with 24.0% yield and example 142 (LC-MS (Method 1): t_{R}=6.15 min; m/z=353 (MH⁺)) with 10.2% yield.

### Example 143

### 6-[(3R)-3-(Methylamino)pyrrolidin-1-yl]-N⁴-phenylpyrimidine-2,4-diamine

A mixture of the compound obtained in reference example 8 (100 mg, 0.305 mmol), in a dioxane/HCl_{(g)} solution (3 mL) was stirred 15 min at room temperature. It was concentrated to dryness and the resulting residue was suspended in EtOH (4 mL). Aniline (0.084 mL, 0.91 mmol) was added and the mixture was irradiated in a multimode microwave at 120° C. for 30 min. It was allowed to cool and 1 mL of a solution of NH₃ (g) in MeOH was added. The solvents were evaporated and the residue was purified by chromatography on silica gel (Biotage cartridge Si Flash) using AcOEt/MeOH/NH₃ mixtures of increasing polarity as eluent, to afford 86 mg of the title compound (yield: 92%).

LC-MS (Method 1): t_{R}=4.59 min; m/z=285 (MH⁺).

### Examples 144-182

Following a similar procedure to that described in example 143, but using the corresponding starting materials in each case, the following compounds were obtained:

| | | | Method | t_{R} | m/z |
|---|---|---|---|---|---|
| Example | Name | Starting materials | (LC-MS) | (min) | (MH⁺) |
| 144 | N⁴-(3-Chlorophenyl)-6-[(3R)-3- | Reference example 8 | 1 | 5.52 | 319 |
| | (methylamino)pyrrolidin-1- | and | | | |
| | yl]pyrimidine-2,4-diamine | 3-chloroaniline | | | |
| 145 | N⁴-(4-Fluorophenyl)-6-[(3R)-3- | Reference example 8 | 1 | 4.79 | 303 |
| | (methylamino)pyrrolidin-1- | and | | | |
| | yl]pyrimidine-2,4-diamine | 4-fluoroaniline | | | |
| 146 | N⁴-(3-Chloro-4-fluorophenyl)-6- | Reference example 8 | 1 | 5.70 | 337 |
| | [(3R)-3-(methylamino)pyrrolidin- | and | | | |
| | 1-yl]pyrimidine-2,4-diamine | 3-chloro-4- | | | |
| | | fluoroaniline | | | |
| 147 | 6-[(3R)-3- | Reference example 8 | 1 | 5.96 | 335 |
| | (Methylamino)pyrrolidin-1-yl]- | and | | | |
| | N⁴-(2-naphthyl)pyrimidine-2,4- | 2-naphthylamine | | | |
| | diamine | | | | |
| 148 | N⁴-(3-Fluorophenyl)-6-[(3R)-3- | Reference example 8 | 1 | 5.14 | 303 |
| | (methylamino)pyrrolidin-1- | and | | | |
| | yl]pyrimidine-2,4-diamine | 3-fluoroaniline | | | |
| 149 | 6-[(3R)-3- | Reference example 8 | 1 | 6.17 | 353 |
| | (Methylamino)pyrrolidin-1-yl]- | and | | | |
| | N⁴-(3- | 3- | | | |
| | trifluoromethylphenyl)pyrimidine- | (trifluoromethyl)aniline | | | |
| | 2,4-diamine | | | | |
| 150 | N⁴-(3,4-Difluorophenyl)-6-[(3R)- | Reference example 8 | 1 | 5.47 | 321 |
| | 3-(methylamino)pyrrolidin-1- | and | | | |
| | yl]pyrimidine-2,4-diamine | 3,4-difluoroaniline | | | |
| 151 | N⁴-(3-Ethynylphenyl)-6-[(3R)-3- | Reference example 8 | 1 | 5.43 | 309 |
| | (methylamino)pyrrolidin-1- | and | | | |
| | yl]pyrimidin-2,4-diamine | 3-ethynylaniline | | | |
| 152 | 3-({2-Amino-6-[(3R)-3- | Reference example 8 | 1 | 3.87 | 301 |
| | (methylamino)pyrrolidin-1- | and | | | |
| | yl]pyrimidin-4-yl}amino)phenol | 3-aminophenol | | | |
| 153 | N⁴-(3-Methoxyphenyl)-6-[(3R)- | Reference example 8 | 1 | 4.91 | 315 |
| | 3-(methylamino)pyrrolidin-1- | and | | | |
| | yl]pyrimidine-2,4-diamine | 3-methoxyaniline | | | |
| 154 | 6-[3-(Methylamino)pyrrolidin-1- | Reference example 9 | 1 | 4.44 | 285 |
| | yl]-N⁴-phenylpyrimidine-2,4- | and | | | |
| | diamine | aniline | | | |
| 155 | 6-[3-(Methylamino)azetidin-1- | Reference example | 1 | 4.68 | 271 |
| | yl]-N⁴-phenylpyrimidine-2,4- | 11 and | | | |
| | diamine | aniline | | | |
| 156 | N⁴-(4-Fluorophenyl)-6-[3- | Reference example | 1 | 4.88 | 289 |
| | (methylamino)azetidin-1- | 11 and | | | |
| | yl]pyrimidine-2,4-diamine | 4-fluoroaniline | | | |
| 157 | N⁴-(3-Chlorophenyl)-6-[3- | Reference example | 1 | 5.40 | 305 |
| | (methylamino)azetidin-1- | 11 and | | | |
| | yl]pyrimidine-2,4-diamine | 3-chloroaniline | | | |
| 158 | N⁴-(3-Chloro-4-fluorophenyl)-6- | Reference example | 1 | 5.68 | 323 |
| | [3-(methylamino)azetidin-1- | 11 and | | | |
| | yl]pyrimidine-2,4-diamine | 3-chloro-4- | | | |
| | | fluoroaniline | | | |
| 159 | 6-[3-(Methylamino)azetidin-1- | Reference example | 1 | 5.20 | 285 |
| | yl]-N⁴-(3- | 11 and | | | |
| | methylphenyl)pyrimidine-2,4- | 3-methylaniline | | | |
| | diamine | | | | |
| 160 | N⁴-(3,4-Difluorophenyl)-6-[3- | Reference example | 1 | 5.22 | 307 |
| | (methylamino)azetidin-1- | 11 and | | | |
| | yl]pyrimidine-2,4-diamine | 3,4-difluoroaniline | | | |
| 161 | N⁴-(3-Fluorophenyl)-6-[3- | Reference example | 1 | 5.00 | 289 |
| | (methylamino)azetidin-1- | 11 and | | | |
| | yl]pyrimidine-2,4-diamine | 3-fluoroaniline | | | |
| 162 | N⁴-(3-Ethynylphenyl)-6-[3- | Reference example | 1 | 5.27 | 295 |
| | (methylamino)azetidin-1- | 11 and | | | |
| | yl]pyrimidine-2,4-diamine | 3-ethynylaniline | | | |
| 163 | N⁴-(4-Fluoro-3-methylphenyl)-6- | Reference example | 1 | 5.40 | 303 |
| | [3-(methylamino)azetidin-1- | 11 and | | | |
| | yl]pyrimidine-2,4-diamine | 4-fluoro-3- | | | |
| | | methylaniline | | | |
| 164 | 6-[3-(Ethylamino)azetidin-1-yl]- | Reference example | 1 | 5.40 | 303 |
| | N⁴-(4-fluorophenyl)pyrimidine- | 12 and | | | |
| | 2,4-diamine | 4-fluoroaniline | | | |
| 165 | 6-[3-(Ethylamino)azetidin-1-yl]- | Reference example | 1 | 5.11 | 285 |
| | N⁴-phenylpyrimidine-2,4- | 12 and | | | |
| | diamine | aniline | | | |
| 166 | N⁴-(3-Chlorophenyl)-6-[3- | Reference example | 1 | 5.86 | 319 |
| | (ethylamino)azetidin-1- | 12 and | | | |
| | yl]pyrimidine-2,4-diamine | 3-chloroaniline | | | |
| 167 | N⁴-(3-Chloro-4-fluorophenyl)-6- | Reference example | 1 | 6.10 | 337 |
| | [3-(ethylamino)azetidin-1- | 12 and | | | |
| | yl]pyrimidine-2,4-diamine | 3-chloro-4- | | | |
| | | fluoroaniline | | | |
| 168 | 6-[3-(Ethylamino)azetidin-1-yl]- | Reference example | 1 | 5.64 | 299 |
| | N⁴-(3-methylphenyl)pyrimidine- | 12 and | | | |
| | 2,4-diamine | 3-methylaniline | | | |
| 169 | N⁴-(3,4-Difluorophenyl)-6-[3- | Reference example | 1 | 5.72 | 321 |
| | (ethylamino)azetidin-1- | 12 and | | | |
| | yl]pyrimidine-2,4-diamine | 3,4-difluoroaniline | | | |
| 170 | 6[3-(Ethylamino)azetidin-1-yl]- | Reference example | 1 | 5.57 | 303 |
| | N⁴-(3-fluorophenyl)pyrimidine- | 12 and | | | |
| | 2,4-diamine | 3-fluoroaniline | | | |
| 171 | 6-[(3R)-3-Aminopyrrolidin-1-yl]- | Reference example | 1 | 4.47 | 289 |
| | N⁴-(4-fluorophenyl)pyrimidine- | 10 and | | | |
| | 2,4-diamine | 4-fluoroaniline | | | |
| 172 | 6-[(3R)-3-Aminopyrrolidin-1-yl]- | Reference example | 1 | 5.36 | 305 |
| | N⁴-(3-chlorophenyl)pyrimidine- | 10 and | | | |
| | 2,4-diamine | 3-chloroaniline | | | |
| 173 | 6-[3-(Dimethylamino)pyrrolidin- | Reference example | 1 | 5.45 | 299 |
| | 1-yl]-N⁴-phenylpyrimidine-2,4- | 13 and | | | |
| | diamine | aniline | | | |
| 174 | 6-[3-(Dimethylamino)pyrrolidin- | Reference example | 1 | 5.36 | 317 |
| | 1-yl]-N⁴-(4- | 13 and | | | |
| | fluorophenyl)pyrimidine-2,4- | 4-fluoroaniline | | | |
| | diamine | | | | |
| 175 | N⁴-(3-Chlorophenyl)-6-[3- | Reference example | 1 | 6.38 | 333 |
| | (dimethylamino)pyrrolidin-1- | 13 and | | | |
| | yl]pyrimidine-2,4-diamine | 3-chloroaniline | | | |
| 176 | 6-(Octahydro-6H-pyrrolo[3,4- | Reference example | 1 | 5.10 | 311 |
| | b]pyridin-6-yl)-N⁴- | 16 and | | | |
| | phenylpyrimidine-2,4-diamine | aniline | | | |
| 177 | N⁴-(4-Fluorophenyl)-6- | Reference example | 1 | 5.33 | 329 |
| | (octahydro-6H-pyrrolo[3,4- | 16 and | | | |
| | b]pyridin-6-yl)pyrimidine-2,4- | 4-fluoroaniline | | | |
| | diamine | | | | |
| 178 | 6-(4-Aminopiperidin-1-yl)-N⁴-(4- | Reference example | 1 | 4.70 | 303 |
| | fluorophenyl)pyrimidine-2,4- | 15 and | | | |
| | diamine | 4-fluoroaniline | | | |
| 179 | 6-(3-Aminopiperidin-1-yl)-N⁴-(4- | Reference example | 1 | 5.12 | 303 |
| | fluorophenyl)pyrimidine-2,4- | 14 and | | | |
| | diamine | 4-fluoroaniline | | | |
| 180 | 6-[(3S)-3- | Reference example | 1 | 4.66 | 285 |
| | (Methylamino)pyrrolidin-1-yl]- | 18 and | | | |
| | N⁴-phenylpyrimidine-2,4- | aniline | | | |
| | diamine | | | | |
| 181 | N⁴-(4-Fluorophenyl)-6-[(3S)-3- | Reference example | 1 | 4.84 | 303 |
| | (methylamino)pyrrolidin-1- | 18 and | | | |
| | yl]pyrimidine-2,4-diamine | 4-fluoroaniline | | | |
| 182 | N⁴-(3-Chlorophenyl)-6-[(3S)-3- | Reference example | 1 | 5.55 | 319 |
| | (methylamino)pyrrolidin-1- | 18 and | | | |
| | yl]pyrimidine-2,4-diamine | 3-chloroaniline | | | |

### Example 183

### N⁴-Benzyl-6-[(3R)-3-(methylamino)pyrrolidin-1-yl]pyrimidine-2,4-diamine

The compound obtained in reference example 8 (150 mg, 0.458 mmol) and benzylamine (1 mL) were introduced into a pressure tube and the mixture was heated at 150° C. for 18 hours. The reaction was filtered and the filtrate was evaporated to dryness. The crude product obtained was purified by reverse phase chromatography (HPLC preparative), using mixtures of AcN/NH₄HCO₃ 75 mM as eluent to afford 102 mg of tert-butyl {(3R)-1-[2-amino-6-(benzylamino)pyrimidin-4-yl]pyrrolidin-3-yl}methylcarbamate. Then, a 4M dioxane/HCl_{(g)} solution (2 mL) was added to 90 mg of this intermediate and the mixture was stirred for 18 hours at room temperature. The solvents were evaporated and the residue was partitioned between CH₂Cl₂ and solution of 0.5N NaOH. The phases were separated and the organic phase was dried over Na₂SO₄ and concentrated to dryness to afford 30 mg of the title compound (yield: 46%).

LC-MS (Method 1): t_{R}=4.74 min; m/z=299 (MH⁺).

### Examples 184-186

Following a similar procedure to that described in example 183, but using the corresponding starting materials in each case, the following compounds were obtained:

| | | | Method | | m/z |
|---|---|---|---|---|---|
| Example | Name | Starting materials | (LC-MS) | t_{R} (min) | (MH⁺) |
| 184 | N⁴-Benzyl-6-[3- | Reference example 9 | 1 | 4.84 | 299 |
| | (methylamino)pyrrolidin-1- | and | | | |
| | yl]pyrimidine-2,4-diamine | benzylamine | | | |
| 185 | 2-Amino-4-((1S)-1- | Reference example 3 | 1 | 4.62 | 299 |
| | phenylethylamino)-6- | and | | | |
| | (piperazin-1-yl)pyrimidine | (S)-(−)-α- | | | |
| | | methylbenzylamine | | | |
| 186 | 2-Amino-6-([1,4]diazepan-1- | Reference example 4 | 1 | 4.69 | 313 |
| | yl)-4-((1S)-1- | and | | | |
| | phenylethylamino)pyrimidine | (S)-(−)-α- | | | |
| | | methylbenzylamine | | | |

### Example 187

N⁴-(2-Fluorophenyl)-6-[(3R)-3-(methylamino)pyrrolidin-1-yl]pyrimidine-2,4-diamine

### (a) tert-Butyl {(3R)-1-[2-(2,5-dimethylpyrrol-1-yl)-6-(2-fluoro-phenylamino)pyrimidin-4-yl]-pyrrolidin-3-yl}methylcarbamate

A mixture of the compound obtained in reference example 23 (150 mg, 0.38 mmol), toluene (2 mL), BINAP (9.48 mg, 0.0152 mmol), Na^{t}BuO (91.5 mg, 0.95 mmol), Pd(OAc)₂ (3.41 mg, 0.0152 mmol) and 2-fluoroaniline (0.073 mL, 0.76 mmol) were introduced into a Schlenk flask. The flask was cycled three times argon/vacuum and the resulting mixture was heated at 105° C. for 18 hours. The reaction was filtered through Celite and the filtrate was evaporated to dryness. The crude product obtained was chromatographed on silica gel (Biotage cartridge Si Flash) using hexane/AcOEt mixtures of increasing polarity as eluent, to afford 84 mg of the desired compound as an oil.

### (b) tert-Butyl {(3R)-1-[2-amino-6-(2-fluoro-phenylamino)pyrimidin-4-yl]pyrrolidin-3-yl}methylcarbamate

The compound obtained above was introduced into a pressure tube together with EtOH (2 mL), H₂O (1 mL), hydroxylamine hydrochloride (121 mg, 1.75 mmol) and Et₃N (0.121 mL, 0.87 mmol) and was heated at 100° C. for 18 hours. The reaction mixture was allowed to cool and then was concentrated to dryness and partitioned between AcOEt and saturated solution of NaHCO₃. The organic phase was separated, dried over Na₂SO₄ and then it was concentrated to dryness to afford 80 mg of the desired compound.

LC-MS (Method 1): t_{R}=7.64 min; m/z=403 (MH⁺)

### (c) Title Compound

To a solution of the compound obtained above in dioxane (1 mL), a 4M dioxane/HCl_{(g)} solution (2 mL) was added and it was stirred at room temperature for 18 hours. The solvents were evaporated and the residue was partitioned between AcOEt and H₂O. A solution of NaOH 3N was then added to reach pH=9 and the aqueous phase was extracted with CH₂Cl₂. The organic phase was dried over Na₂SO₄ and concentrated to dryness to afford a crude product which was chromatographed on silica gel using AcOEt/MeOH mixtures of increasing polarity as eluent, to afford 23 mg of the title compound (yield for the three steps: 20%).

LC-MS (Method 3): t_{R}=4.52 min; m/z=303 (MH⁺).

### Examples 188-196

Following a similar procedure to that described in example 187, but using the corresponding starting materials in each case, the following compounds were obtained:

| | | | Method | t_{R} | m/z |
|---|---|---|---|---|---|
| Example | Name | Starting materials | (LC-MS) | (min) | (MH⁺) |
| 188 | 6-[(3R)-3- | Reference example | 3 | 5.11 | 299 |
| | (Methyamino)pyrrolidin-1-yl]-N⁴- | 23 and | | | |
| | (3-methylphenyl)pyrimidine-2,4- | 3-methylaniline | | | |
| | diamine | | | | |
| 189 | N⁴-(2,4-Difluorophenyl)-6-[(3R)- | Reference example | 3 | 4.74 | 321 |
| | 3-(methylamino)pyrrolidin-1- | 23 and | | | |
| | yl]pyrimidine-2,4-diamine | 2,4-difluoroaniline | | | |
| 190 | N⁴-(3-Fluoro-2-methylphenyl)-6- | Reference example | 1 | 5.21 | 317 |
| | [(3R)-3-(methylamino)pyrrolidin- | 23 and | | | |
| | 1-yl]pyrimidine-2,4-diamine | 3-fluoro-2- | | | |
| | | methylaniline | | | |
| 191 | 2-Amino-6-([1,4]diazepan-1-yl)- | Reference example | 1 | 4.45 | 321 |
| | 4-(2,4- | 20 and | | | |
| | difluorophenylamino)pyrimidine | 2,4-difluoroaniline | | | |
| 192 | 2-Amino-6-(4-methyl- | Reference example | 1 | 6.45 | 353 |
| | [1,4]diazepan-1-yl)-4-(2,3,5- | 22 and | | | |
| | trifluorophenylamino)pyrimidine | 2,3,5-trifluoroaniline | | | |
| 193 | 2-Amino-4-(3-chloro-2- | Reference example | 1 | 6.34 | 351 |
| | fluorophenylamino)-6-(4-methyl- | 22 and | | | |
| | [1,4]diazepan-1-yl)pyrimidine | 3-chloro-2- | | | |
| | | fluoroaniline | | | |
| 194 | 2-Amino-4-(2-fluoro-5- | Reference example | 1 | 5.82 | 331 |
| | methylphenylamino)-6-(4- | 22 and | | | |
| | methyl-[1,4]diazepan-1- | 2-fluoro-5- | | | |
| | yl)pyrimidine | methylaniline | | | |
| 195 | N⁴-(3-Chlorophenyl)-6-[(3R)-3- | Reference example | 1 | 5.96 | 333 |
| | (ethylamino)pyrrolidin-1- | 24 and | | | |
| | yl]pyrimidine-2,4-diamine | 3-chloroaniline | | | |
| 196 | 6-[(3R)-3-(Ethylamino)pyrrolidin- | Reference example | 1 | 4.96 | 299 |
| | 1-yl]-N⁴-phenylpyrimidine-2,4- | 24 and | | | |
| | diamine | aniline | | | |

### Example 197

### 6-[(3R)-3-Aminopyrrolidin-1-yl]-N⁴-phenylpyrimidine-2,4-diamine

### (a) tert-Butyl[1-(2-amino-6-phenylamino-pyrimidin-4-yl)pyrrolidin-3-yl]-carbamate

The compound obtained in reference example 25 (107 mg, 0.49 mmol), tert-butyl (3R)-pyrrolidin-3-ylcarbamate (100 mg, 0.54 mmol), n-BuOH (3.8 mL) and DIEA (0.09 mL, 0.51 mmol) were reacted in a pressure tube. The mixture was heated at 120° C. for 24 hours and then was concentrated to dryness. The crude product obtained was chromatographed on silica gel (Biotage cartridge Si Flash) using AcOEt as eluent, to afford 38 mg of the desired compound.

### (b) Title Compound

The compound obtained above was treated with 4M dioxane/HCl_{(g)} solution (3 mL) and was stirred at room temperature for 18 hours. The solvents were evaporated and the residue was partitioned between AcOEt and H₂O. A solution of 1N NaOH was then added to reach pH=7-8 and the aqueous phase was extracted with AcOEt. The organic phase was dried over Na₂SO₄ and concentrated to dryness to afford 11 mg of the title compound (yield for the two steps: 8%).

LC-MS (Method 1): t_{R}=4.10 min; m/z=271 (MH⁺).

### Example 198

### 6-[(3S)-3-Aminopyrrolidin-1-yl]-N⁴-phenylpyrimidine-2,4-diamine

Following a similar procedure to that described in example 197, but using tert-butyl (3S)-pyrrolidin-3-ylcarbamate instead of tert-butyl (3R)-pyrrolidin-3-ylcarbamate, the desired compound was obtained (yield: 2%).

LC-MS (Method 1): t_{R}=4.41 min; m/z=271 (MH⁺).

### Example 199

### 2-Amino-4-(3-ethynylphenylamino)-6-(4-methyl-[1,4]diazepan-1-yl)pyrimidine

The compound obtained in reference example 1 (70 mg, 0.28 mmol), 3-ethynylaniline (0.091 mL, 0.86 mmol) and EtOH (5 mL) were introduced into a pressure tube. The mixture was heated at 90° C. for 64 hours and then was concentrated to dryness. The residue was partitioned between AcOEt and a solution of 1N NaOH. The organic phase was dried over Na₂SO₄ and concentrated to dryness. The crude product obtained was chromatographed on silica gel (Biotage cartridge Si Flash) using CHCl₃/MeOH mixtures of increasing polarity as eluent, to afford 52 mg of the title compound (yield: 55%).

LC-MS (Method 1): t_{R}=5.68 min; m/z=323 (MH⁺).

### Example 200

### 2-Amino-6-(4-methylpiperazin-1-yl)-4-((1S)-1-phenylethylamino)pyrimidine

The compound obtained in reference example 2 (100 mg, 0.439 mmol) and (S)-(−)-α-methylbenzylamine (1 mL, 7.85 mmol) were introduced into a pressure tube. The mixture was heated at 180° C. for 18 hours and then was concentrated to dryness. The residue was partitioned between CH₂Cl₂ and a solution of 1N NaOH. The organic phase was dried over Na₂SO₄ and concentrated to dryness. The crude product obtained was chromatographed on silica gel using CH₂Cl₂/MeOH mixtures of increasing polarity as eluent, to afford 133 mg of the title compound (yield: 97%).

LC-MS (Method 1): t_{R}=5.33 min; m/z=313 (MH⁺).

### Example 201

### 2-Amino-4-[(2-methoxyphenylmethyl)amino]-6-(4-methylpiperazin-1-yl)pyrimidine

Following a similar procedure to that described in example 200, but using 2-methoxybenzylamine instead of (S)-(−)-α-methylbenzylamine, the desired compound was obtained (yield: 40%).

LC-MS (Method 1): t_{R}=5.41 min; m/z=329 (MH⁺).

### Example 202

### 2-Amino-4-[(4-fluorophenylmethyl)amino]-6-(4-methylpiperazin-1-yl)pyrimidine

Following a similar procedure to that described in example 200, but using 4-fluorobenzylamine instead of (S)-(−)-α-methylbenzylamine, the desired compound was obtained (yield: 54%).

LC-MS (Method 1): t_{R}=5.3 min; m/z=317 (MH⁺).

### Example 203

### Biological Assay

### Binding Competition Assay of [³H]-Histamine to Human Histamine H₄ Receptor

The activity of the compounds of the invention against the H₄ receptor can be tested using the following binding assay.

Membrane extracts prepared from a stable CHO recombinant cell line which express the human histamine H₄ receptor are used.

Test compounds are incubated at the selected concentration in duplicate, with 10 nM [³H]-histamine and 15 μg membranes extract in a total volume of 250 μL 50 mM Tris-HCl, pH 7.4, 1.25 mM EDTA at 25° C. for 60 minutes. The non-specific binding is defined in the presence of 100 μM unlabeled histamine. The reaction is stopped by filtration using a vacuum collector (Multiscreen Millipore) in 96 well plates (MultiScreen HTS Millipore) which have been previously soaked in a 0.5% polyethylenimine solution at 0° C. for 2 hours. Subsequently, the plates are washed with 50 mM Tris (pH 7.4), 1.25 mM EDTA at 0° C. and filters are dried during 1 hour at 50-60° C., before adding the scintillation liquid to determine bound radioactivity by using a betaplate scintillation counter.

1-13. (canceled)

14. A compound of formula I 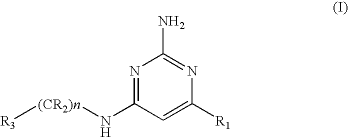
wherein:
R₁ is a group of formula (a): 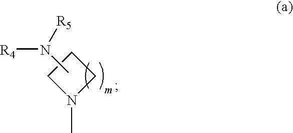
R₂ is chosen from a hydrogen atom and C₁₋₄ alkyl groups;
R₃ is a phenyl group, optionally fused to a 5- or 6-membered aromatic, saturated, or partially unsaturated ring, which can be carbocyclic or heterocyclic with 1 or 2 heteroatoms chosen from N, O, and S, and wherein R₃ is optionally substituted with one or more substituents R₈;
R₄ and R₅ are each independently chosen from a hydrogen atom and C₁₋₄ alkyl groups;
each instance of R₈ is independently chosen from halogen atoms, C₁₋₄ alkyl, —OH, C₁₋₄ alkoxy, C14 alkylthio, C₁₋₄ haloalkyl, C₁₋₄ haloalkoxy, —COR₉, —CO₂R₉, —CONR₉R₉, —NR₉R₉, —NHCOR₁₀, —CN, C₂₋₄ alkynyl, and —CH₂OH groups, and additionally one of the substituents R₈ can be a phenyl group optionally substituted with one or more substituents each independently chosen from halogen atoms, C₁₋₄ alkyl, —OH, C₁₋₄ alkoxy, C₁₋₄ alkylthio, C₁₋₄ haloalkyl, C₁₋₄ haloalkoxy, —COR₉, —CO₂R₉, —CONR₉R₉, —NR₉R₉, —NHCOR₁₀, —CN, C₂₋₄ alkynyl, and —CH₂OH groups;
R₉ is chosen from a hydrogen atom and C₁₋₄ alkyl groups;
R₁₀ is chosen from C₁₋₄ alkyl groups;
m is 1 or 2; and
n is 0 or 1;
or a salt thereof.

15. The compound according to claim 14, wherein n is 0.

16. The compound according to claim 14, wherein R₂ is chosen from a hydrogen atom and a methyl group.

17. The compound according to claim 14, wherein R₃ is chosen from phenyl and naphthyl groups, optionally substituted with one or more substituents R₈.

18. The compound according to claim 17, wherein R₃ is a phenyl group optionally substituted with one or more substituents R₈.

19. The compound according to claim 14, wherein each instance of R₈ is independently chosen from halogen atoms, C₁₋₄ alkyl, —OH, C₁₋₄ alkoxy, C₁₋₄ haloalkyl, C₁₋₄ haloalkoxy, —CN, and C₂₋₄ alkynyl groups, and additionally one of the substituents R₈ can be a phenyl group optionally substituted with one or more substituents chosen from halogen atoms, C₁₋₄ alkyl, —OH, C₁₋₄ alkoxy, C₁₋₄ haloalkyl, C₁₋₄ haloalkoxy, —CN, and C₂₋₄ alkynyl groups.

20. The compound according to claim 19, wherein each instance of R₈ is independently chosen from halogen atoms, C₁₋₄ alkyl, —OH, C₁₋₄ alkoxy, C₁₋₄ haloalkyl, C₁₋₄ haloalkoxy, —CN, and C₂₋₄ alkynyl groups.

21. The compound according to claim 14, wherein R₄ is chosen from a hydrogen atom and C₁₋₂ alkyl groups.

22. The compound according to claim 14, wherein R₅ is chosen from a hydrogen atom and C₁₋₂ alkyl groups.

23. The compound according to claim 14, wherein R₄ is a hydrogen atom and R₅ is chosen from C₁₋₂ alkyl groups.

24. The compound according to claim 14, wherein R₄ is a hydrogen atom and R₅ is a hydrogen atom.

25. The compound according to claim 14, wherein R₄ is a methyl group and R₅ is a methyl group.

26. The compound according to claim 14, wherein n is 0 and R₃ is a phenyl group optionally substituted with one or more substituents R₈.

27. The compound according to claim 26, wherein each instance of R₈ is independently chosen from halogen atoms, C₁₋₄ alkyl, —OH, C₁₋₄ alkoxy, C₁₋₄ haloalkyl, C₁₋₄ haloalkoxy, —CN, and C₂₋₄ alkynyl groups.

28. The compound according to claim 14 chosen from:
2-Amino-4-(2,4-difluorophenylamino)-6-(3-(methylamino)azetidin-1-yl)pyrimidine;
2-Amino-6-(3-(methylamino)azetidin-1-yl)-4-(3-(trifluoromethyl)phenylamino)pyrimidine;
2-Amino-4-(2-fluorophenylamino)-6-(3-(methylamino)azetidin-1-yl)pyrimidine;
2-Amino-4-(4-fluoro-3-methylphenylamino)-6-((3R)-3-(methylamino)pyrrolidin-1-yl)pyrimidine;
2-Amino-4-(3-ethylphenylamino)-6-((3R)-3-(methylamino)pyrrolidin-1-yl)pyrimidine;
2-Amino-6-((3R)-3-(methylamino)pyrrolidin-1-yl)-4-(3,4,5-trifluorophenylamino)pyrimidine;
6-(3-(Methylamino)azetidin-1-yl)-N⁴-(3,4,5-trifluorophenyl)pyrimidine-2,4-diamine;
N⁴-(3-Chloro-4-fluorophenyl)-6-[(3S)-3-(methylamino)pyrrolidin-1-yl]pyrimidine-2,4-diamine;
6-[(3S)-3-(methylamino)pyrrolidin-1-yl]-N⁴-m-tolylpyrimidine-2,4-diamine;
N⁴-(3,4-Difluorophenyl)-6-[(3S)-3-(methylamino)pyrrolidin-1-yl]pyrimidine-2,4-diamine;
N⁴-(3-Trifluoromethylphenyl)-6-[(3S)-3-(methylamino)pyrrolidin-1-yl]pyrimidine-2,4-diamine;
3-[2-Amino-6-[(3R)-3-(methylamino)pyrrolidin-1-yl]pyrimidin-4-ylamino]-2-methylphenol;
N⁴-(4-Fluoro-3-methoxyphenyl)-6-[(3R)-3-(methylamino)pyrrolidin-1-yl]pyrimidine-2,4-diamine;
N⁴-(2,4-Difluoro-3-methoxyphenyl)-6-[(3R)-3-(methylamino)pyrrolidin-1-yl]pyrimidine-2,4-diamine;
N⁴-(2-Fluorophenyl)-6-[(3S)-3-(methylamino)pyrrolidin-1-yl]pyrimidine-2,4-diamine;
N⁴-(3-Fluorophenyl)-6-[(3S)-3-(methylamino)pyrrolidin-1-yl]pyrimidine-2,4-diamine;
6-[(3R)-3-aminopyrrolidin-1-yl]-N⁴-m-tolylpyrimidine-2,4-diamine;
6-[(3R)-3-aminopyrrolidin-1-yl]-N⁴-(3-chloro-4-fluorophenyl)pyrimidine-2,4-diamine;
6-[(3R)-3-aminopyrrolidin-1-yl]-N⁴-(2-fluorophenyl)pyrimidine-2,4-diamine;
6-[(3R)-3-aminopyrrolidin-1-yl]-N⁴-(4-fluoro-3-methylphenyl)pyrimidine-2,4-diamine;
6-[(3R)-3-aminopyrrolidin-1-yl]-N⁴-(3,4-difluorophenyl)pyrimidine-2,4-diamine;
6-[(3R)-3-aminopyrrolidin-1-yl]-N⁴-(3-fluorophenyl)pyrimidine-2,4-diamine;
3-[2-Amino-6-(3-(methylamino)azetidin-1-yl)-pyrimidin-4-ylamino]phenol;
N⁴-(3-Methoxyphenyl)-6-(3-(methylamino)azetidin-1-yl)-pyrimidine-2,4-diamine;
6-(3-(Methylamino)azetidin-1-yl)-N⁴-naphthalen-2-ylpyrimidine-2,4-diamine;
3-[2-Amino-6-(3-(methylamino)azetidin-1-yl)-pyrimidin-4-ylamino]benzonitrile;
N⁴-(4-Fluoro-3-methoxyphenyl)-6-(3-(methylamino)azetidin-1-yl)-pyrimidine-2,4-diamine;
5-[2-Amino-6-(3-(methylamino)azetidin-1-yl)-pyrimidin-4-ylamino]-2-fluoro-benzonitrile;
N⁴-(3-Ethylphenyl)-6-(3-(methylamino)azetidin-1-yl)-pyrimidine-2,4-diamine;
N⁴-(2,4-Difluoro-3-methoxyphenyl)-6-(3-(methylamino)azetidin-1-yl)-pyrimidine-2,4-diamine;
N⁴-(2,3-Difluorophenyl)-6-(3-(methylamino)azetidin-1-yl)-pyrimidine-2,4-diamine;
2-Amino-6-(3-(methylamino)azetidin-1-yl)-4-(2-tolylamino)pyrimidine;
2-Amino-4-(3-chloro-2-fluorophenylamino)-6-((3R)-3-(methylamino)pyrrolidin-1-yl)pyrimidine;
2-Amino-4-(2,3-difluorophenylamino)-6-((3R)-3-(methylamino)pyrrolidin-1-yl)pyrimidine;
2-Amino-4-(4-fluoro-2-methylphenylamino)-6-((3R)-3-(methylamino)pyrrolidin-1-yl)pyrimidine;
2-Amino-4-(3-chloro-2-methylphenylamino)-6-((3R)-3-(methylamino)pyrrolidin-1-yl)pyrimidine;
2-Amino-4-(2-chloro-4-fluorophenylamino)-6-((3R)-3-(methylamino)pyrrolidin-1-yl)pyrimidine;
N⁴-(3-Chloro-2-fluorophenyl)-6-(3-(methylamino)azetidin-1-yl)pyrimidine-2,4-diamine;
N⁴-(3-Fluoro-2-methylphenyl)-6-(3-(methylamino)azetidin-1-yl)pyrimidine-2,4-diamine;
6-(3-(Methylamino)azetidin-1-yl)-N⁴-(2,3,4-trifluorophenyl)pyrimidine-2,4-diamine;
N⁴-(4-Fluoro-2-methylphenyl)-6-(3-(methylamino)azetidin-1-yl)pyrimidine-2,4-diamine;
N⁴-(2-Chloro-4-fluorophenyl)-6-(3-(methylamino)azetidin-1-yl)pyrimidine-2,4-diamine;
6-[(3R)-3-(Methylamino)pyrrolidin-1-yl]-N⁴-(2,3,4-trifluorophenyl)pyrimidine-2,4-diamine;
N⁴-(2,3-Dichlorophenyl)-6-[(3R)-3-(methylamino)pyrrolidin-1-yl]pyrimidine-2,4-diamine;
N⁴-(2,3-Dimethylphenyl)-6-[(3R)-3-(methylamino)pyrrolidin-1-yl]pyrimidine-2,4-diamine;
6-[(3R)-3-(Dimethylamino)pyrrolidin-1-yl]-N⁴-m-tolyl-pyrimidine-2,4-diamine;
N⁴-(3-Chloro-4-fluorophenyl)-6-[(3R)-3-(dimethylamino)pyrrolidin-1-yl]pyrimidine-2,4-diamine;
N⁴-(3,4-Difluorophenyl)-6-[(3R)-3-(dimethylamino)pyrrolidin-1-yl]pyrimidine-2,4-diamine;
N⁴-(4-Fluoro-3-methylphenyl)-6-[(3R)-3-(dimethylamino)pyrrolidin-1-yl]pyrimidine-2,4-diamine;
N⁴-(3-Chloro-2-fluorophenyl)-6-[(3R)-3-(dimethylamino)pyrrolidin-1-yl]pyrimidine-2,4-diamine;
6-[(3R)-3-Aminopyrrolidin-1-yl]-N⁴-(3-ethynylphenyl)pyrimidine-2,4-diamine;
6-[(3R)-3-Aminopyrrolidin-1-yl]-N⁴-(3,4,5-trifluorophenyl)pyrimidine-2,4-diamine;
6-[(3R)-3-Aminopyrrolidin-1-yl]-N⁴-(4-fluoro-2-methylphenyl)pyrimidine-2,4-diamine;
6-[(3R)-3-Aminopyrrolidin-1-yl]-N⁴-(3-trifluoromethylphenyl)pyrimidine-2,4-diamine;
6-[(3R)-3-(Dimethylamino)pyrrolidin-1-yl]-N⁴-phenylpyrimidine-2,4-diamine;
6-[(3R)-3-(Dimethylamino)pyrrolidin-1-yl]-N⁴-(4-fluorophenyl)pyrimidine-2,4-diamine;
N⁴-(3-Chlorophenyl)-6-[(3R)-3-(dimethylamino)pyrrolidin-1-yl]pyrimidine-2,4-diamine;
6-[(3R)-3-(Dimethylamino)pyrrolidin-1-yl]-N⁴-(2-fluorophenyl)pyrimidine-2,4-diamine;
6-[(3R)-3-(Dimethylamino)pyrrolidin-1-yl]-N⁴-(3-fluorophenyl)pyrimidine-2,4-diamine;
6-[(3R)-3-(Methylamino)pyrrolidin-1-yl]-N⁴-phenylpyrimidine-2,4-diamine;
N⁴-(3-Chlorophenyl)-6-[(3R)-3-(methylamino)pyrrolidin-1-yl]pyrimidine-2,4-diamine;
N⁴-(4-Fluorophenyl)-6-[(3R)-3-(methylamino)pyrrolidin-1-yl]pyrimidine-2,4-diamine;
N⁴-(3-Chloro-4-fluorophenyl)-6-[(3R)-3-(methylamino)pyrrolidin-1-yl]pyrimidine-2,4-diamine;
6-[(3R)-3-(Methylamino)pyrrolidin-1-yl]-N⁴-(2-naphthyl)pyrimidine-2,4-diamine;
N⁴-(3-Fluorophenyl)-6-[(3R)-3-(methylamino)pyrrolidin-1-yl]pyrimidine-2,4-diamine;
6-[(3R)-3-(Methylamino)pyrrolidin-1-yl]-N⁴-(3-trifluoromethylphenyl)pyrimidine-2,4-diamine;
N⁴-(3,4-Difluorophenyl)-6-[(3R)-3-(methylamino)pyrrolidin-1-yl]pyrimidine-2,4-diamine;
N⁴-(3-Ethynylphenyl)-6-[(3R)-3-(methylamino)pyrrolidin-1-yl]pyrimidin-2,4-diamine;
3-({2-Amino-6-[(3R)-3-(methylamino)pyrrolidin-1-yl]pyrimidin-4-yl}amino)phenol;
N⁴-(3-Methoxyphenyl)-6-[(3R)-3-(methylamino)pyrrolidin-1-yl]pyrimidine-2,4-diamine;
6-[3-(Methylamino)pyrrolidin-1-yl]-N⁴-phenylpyrimidine-2,4-diamine;
6-[3-(Methylamino)azetidin-1-yl]-N⁴-phenylpyrimidine-2,4-diamine;
N⁴-(4-Fluorophenyl)-6-[3-(methylamino)azetidin-1-yl]pyrimidine-2,4-diamine;
N⁴-(3-Chlorophenyl)-6-[3-(methylamino)azetidin-1-yl]pyrimidine-2,4-diamine;
N⁴-(3-Chloro-4-fluorophenyl)-6-[3-(methylamino)azetidin-1-yl]pyrimidine-2,4-diamine;
6-[3-(Methylamino)azetidin-1-yl]-N⁴-(3-methylphenyl)pyrimidine-2,4-diamine;
N⁴-(3,4-Difluorophenyl)-6-[3-(methylamino)azetidin-1-yl]pyrimidine-2,4-diamine;
N⁴-(3-Fluorophenyl)-6-[3-(methylamino)azetidin-1-yl]pyrimidine-2,4-diamine;
N⁴-(3-Ethynylphenyl)-6-[3-(methylamino)azetidin-1-yl]pyrimidine-2,4-diamine;
N⁴-(4-Fluoro-3-methylphenyl)-6-[3-(methylamino)azetidin-1-yl]pyrimidine-2,4-diamine;
6-[3-(Ethylamino)azetidin-1-yl]-N⁴-(4-fluorophenyl)pyrimidine-2,4-diamine;
6-[3-(Ethylamino)azetidin-1-yl]-N⁴-phenylpyrimidine-2,4-diamine;
N⁴-(3-Chlorophenyl)-6-[3-(ethylamino)azetidin-1-yl]pyrimidine-2,4-diamine;
N⁴-(3-Chloro-4-fluorophenyl)-6-[3-(ethylamino)azetidin-1-yl]pyrimidine-2,4-diamine;
6-[3-(Ethylamino)azetidin-1-yl]-N⁴-(3-methylphenyl)pyrimidine-2,4-diamine;
N⁴-(3,4-Difluorophenyl)-6-[3-(ethylamino)azetidin-1-yl]pyrimidine-2,4-diamine;
6-[3-(Ethylamino)azetidin-1-yl]-N⁴-(3-fluorophenyl)pyrimidine-2,4-diamine;
6-[(3R)-3-Aminopyrrolidin-1-yl]-N⁴-(4-fluorophenyl)pyrimidine-2,4-diamine;
6-[(3R)-3-Aminopyrrolidin-1-yl]-N⁴-(3-chlorophenyl)pyrimidine-2,4-diamine;
6-[3-(Dimethylamino)pyrrolidin-1-yl]-N⁴-phenylpyrimidine-2,4-diamine;
6-[3-(Dimethylamino)pyrrolidin-1-yl]-N⁴-(4-fluorophenyl)pyrimidine-2,4-diamine;
N⁴-(3-Chlorophenyl)-6-[3-(dimethylamino)pyrrolidin-1-yl]pyrimidine-2,4-diamine;
6-[(3S)-3-(Methylamino)pyrrolidin-1-yl]-N⁴-phenylpyrimidine-2,4-diamine;
N⁴-(4-Fluorophenyl)-6-[(3S)-3-(methylamino)pyrrolidin-1-yl]pyrimidine-2,4-diamine;
N⁴-(3-Chlorophenyl)-6-[(3S)-3-(methylamino)pyrrolidin-1-yl]pyrimidine-2,4-diamine;
N⁴-Benzyl-6-[(3R)-3-(methylamino)pyrrolidin-1-yl]pyrimidine-2,4-diamine;
N⁴-Benzyl-6-[3-(methylamino)pyrrolidin-1-yl]pyrimidine-2,4-diamine;
N⁴-(2-Fluorophenyl)-6-[(3R)-3-(methylamino)pyrrolidin-1-yl]pyrimidine-2,4-diamine;
6-[(3R)-3-(Methylamino)pyrrolidin-1-yl]-N⁴-(3-methylphenyl)pyrimidine-2,4-diamine;
N⁴-(2,4-Difluorophenyl)-6-[(3R)-3-(methylamino)pyrrolidin-1-yl]pyrimidine-2,4-diamine;
N⁴-(3-Fluoro-2-methylphenyl)-6-[(3R)-3-(methylamino)pyrrolidin-1-yl]pyrimidine-2,4-diamine;
N⁴-(3-Chlorophenyl)-6-[(3R)-3-(ethylamino)pyrrolidin-1-yl]pyrimidine-2,4-diamine;
6-[(3R)-3-(Ethylamino)pyrrolidin-1-yl]-N⁴-phenylpyrimidine-2,4-diamine;
6-[(3R)-3-Aminopyrrolidin-1-yl]-N⁴-phenylpyrimidine-2,4-diamine;
6-[(3S)-3-Aminopyrrolidin-1-yl]-N⁴-phenylpyrimidine-2,4-diamine;
and salts thereof.

29. A pharmaceutical composition comprising a compound according to claim 14, or a pharmaceutically acceptable salt thereof, and one or more pharmaceutically acceptable excipients.

30. A method for treating or preventing a pathological condition or disease mediated by the histamine H₄ receptor in a subject in need thereof, which comprises administering to said subject an effective amount of a compound according to claim 14.

31. The method according to claim 30, wherein the pathological condition or disease is chosen from immunological and inflammatory diseases.

32. The method according to claim 30, wherein the pathological condition or disease is chosen from asthma, allergic rhinitis, chronic obstructive pulmonary disease, allergic rhinoconjunctivitis, cutaneous allergic diseases, inflammatory bowel diseases, rheumatoid arthritis, and psoriasis.
